(19) United States
(12) Patent Application Publication
(10) Pub. No.: US 2026/0115239 A1
(43) Pub. Date: Apr. 30, 2026

(54) Nutritional composition comprising urea, non-digestible oligosaccharides and bifidobacteria

(71) Applicant: N.V. Nutricia, Zoetermeer (NL)

(72) Inventors: Cornelus Johannes Petrus van Limpt, Utrecht (NL); Roger Bongers, Utrecht (NL)

(21) Appl. No.: 19/407,063

(22) Filed: Dec. 3, 2025

Related U.S. Application Data
(63) Continuation of application No. PCT/EP2024/068162, filed on Jun. 27, 2024.

(30) Foreign Application Priority Data
Jun. 28, 2023 (EP) 23182145.5

(51) Int. Cl. A61K 35/745; A61K 31/702

(52) U.S. Cl. CPC A61K 35/745; A61K 31/702

(57) ABSTRACT

The invention relates to nutritional compositions for infants and in particular formulae for infants comprising human milk oligosaccharides, specific bifidobacteria, and urea as non-protein nitrogen source for improving the intestinal microbiota.

### FIELD OF THE INVENTION

The present invention relates to nutritional compositions for infants and in particular formulae for infants comprising human milk oligosaccharides, bifiobacteria, and non-protein nitrogen sources for improving the intestinal microbiota.

### BACKGROUND OF THE INVENTION

The human gut harbors a complex microbial ecosystem, the intestinal microbiota, which has been recognized as an essential part of our human physiology. In human adults the intestinal microbiota is considered to be a stable ecosystem, hence the microbial colonization process in early-life, which is heavily intertwined with the maturation of the gastrointestinal tract itself, can be considered as fundamental step in healthy development. Early-life nutrition is a major factor that impacts the developing intestinal microbiota community and breastfeeding infants provides for an optimal intestinal microbiota dominated by Bifidobacterium species. As a consequence, breastfeeding has been associated with a multitude of health benefits related to for example infections, inflammation, allergy, eczema, diarrhea, constipation and so on.

In some cases breastfeeding is inadequate or unsuccessful for medical reasons, or because of a choice not to breastfeed. For such situations infant and follow on formulae have been developed. Commercial infant formulae are commonly used today to provide supplemental or the sole source of nutrition early in life. These formulae comprise a range of nutrients to meet the nutritional needs of the growing infant, and typically include fat, carbohydrate, protein, vitamins, minerals, and other nutrients helpful for optimal infant growth and development. Commercial infant formulae are designed to mimic, as closely as possible, the composition of human milk and at least equally important mimic as good as possible the function of human milk.

In order to positively affect the intestinal microbiota, infant formulae have been developed that include probiotics, hence bifidobacteria themselves, or prebiotics, in particular non-digestible oligosaccharides that promote the growth of intestinal microbiota, or that include both, such as for example is disclosed in WO 2005/110121.

Protein and amino acid supplementation to develop a healthy microbiota ecosystem have also been suggested, such as for instance disclosed in EP 1638418.

Infant formulae are often based on cow's milk. Cow's milk comprises more protein and less non-protein nitrogen (NPN), such as urea, compared to human milk.

In addressing this difference GB 993,719 discloses a specific process to produce an infants' milk which includes the addition of urea to be closer to human milk.

DD 208 542 is concerned with providing more breast milk-like bifidogenic nutrition based on cow's milk by diluting standard formula based on cow's milk to decrease the protein content and supplementing this with lactose and urea.

WO 2015/105616 discloses pediatric nutritional compositions comprising a protein source comprising an intact milk protein and a partially hydrolyzed milk protein, wherein about 5% to about 25% of total nitrogen content of the composition is non-protein nitrogen. The focus of NPN here is on small peptides and amino acids and not on urea.

WO 2020/089463 discloses formulae comprising a combination of urea and non-digestible oligosaccharides, for improved stimulation of the growth of bifidobacteria in the intestinal microbiota of infants or young children.

Walsh et al, 2022, Scientific reports, 12, 4143, discloses a combination of the specific strains B. bifidum R0071, B. infantis R0033, B. infantis M-63 and B breve M-16V to have a synergistic effect when grown on human milk oligosaccharides.

Still there is a need for further improvement of developing a beneficial intestinal microbiota and improving the intestinal microbial resilience in particular early in life in subjects suffering from or being at risk of intestinal microbial dysbiosis.

### SUMMARY OF THE INVENTION

The inventors found that a combination of urea, human milk oligosaccharide selected from fucosyllactoses and sialyllactoses, and a combination of a Bifidobacterium longum ssp infantis that is urease positive with a urease negative Bifidobacterium bifidum that is able to hydrolyze externally fucosyllactose and sialyllactose improved the fermentation by and growth of bifidobacteria in a synergistic manner. This synergy was not observed in the absence of urea. Therefore, a nutritional composition comprising such human milk oligosaccharides, urea and bifidobacteria beneficially further improves the microbiota and related health effects in infants or young children.

### DETAILED DESCRIPTION OF THE INVENTION

The invention thus concerns a nutritional composition comprising urea, non-digestible oligosaccharide, and lactic acid producing bacteria, wherein the non-digestible oligosaccharide comprises one or more human milk oligosaccharides selected from the group consisting of 2′-fucosyllacose (2′-FL), 3-fucosyllactose (3-FL), 3′-sialyllactose (3′-SL), and 6′-sialyllactose (6′-SL), and wherein the lactic acid producing bacteria comprise a strain of Bifidobacterium longum ssp infantis able to express urease, and a strain of Bifidobacterium bifidum able to express at least one extracellular enzyme selected from a fucosidase and a sialidase and not able to express a urease.

### Urea

In the context of the present invention, protein is defined as the sum of protein, peptides and free proteinogenic amino acids. Protein according to the present invention can also be referred to as protein equivalent. Proteinogenic amino acids are the 20 different natural amino acids that can be part of eukaryotic protein and peptides. Non-protein nitrogen (NPN) according to the invention refers to other components than protein equivalent, and that contain nitrogen such as urea, nucleotides, polyamines, some phospholipids, ammonia, ammonium salts, non-proteinogenic amino acids etc.

The most abundant NPN in human milk is urea. In human milk the amount of urea can approximately be 10-13% of total nitrogen, resulting in a concentration of about 15 to 35 mg urea per 100 ml, with an average around 30-35 mg per 100 ml. It has been demonstrated that urea in breast milk can be incorporated in plasma protein by the infant. Furthermore, the content of urea in cow's milk is lower than in human milk. Infant formula manufacturers use casein and whey protein sources that have been prepared by electrodialysis, ultrafiltration or ion-exchange chromatography, rather than whole cow's milk when preparing formulae. Therefore, the nitrogen composition of the protein sources used for manufacturing (modified) cow's milk-based formulae can be quite different and the proportion of NPN, in particular urea, will also vary considerably and can expected to be lower than in natural cow's milk, let alone than in human milk. Methods are known in the art to determine NPN and urea.

The nutritional composition according to the present invention comprises urea. Urea is also known as carbamide, CO(NH₂)₂. Urea is commercially available from various sources in food grade quality, for example from NuGenTec, CA USA, Spectrum chemical, USA, or it can be enriched from cow's milk as disclosed in U.S. Pat. No. 6,506,305. It was found that urea and a urease positive Bifidobacterium longum ssp infantis together with human milk oligosaccharides and Bifidobacterium bifidum have a synergistic effect on growth of bifidobacteria and their production of organic acids.

The nutritional composition preferably comprises at least 1.13 wt % urea based on total protein, more preferably 1.13 to 5.6 wt % urea based on total protein, even more preferably 1.45 to 3.7 wt %, even more preferably 1.5 to 2.6 wt % urea based on total protein. Preferably the nutritional composition comprises at least 0.11 wt % urea based on dry weight of the composition, more preferably 0.11 to 0.55 wt % urea, even more preferably 0.15 to 0.37 wt % urea based on dry weight of the composition. Preferably the nutritional composition comprises at least 15 mg urea per 100 ml, more preferably 15 to 75 mg urea per 100 ml. More preferably the nutritional composition comprises 20 to 60 mg per 100 ml, even more preferably 20 to 50 mg per 100 ml, most preferably 20 to 35 mg urea per 100 ml. Preferably the nutritional composition comprises at least 22 mg urea per 100 kcal, more preferably 22 to 112 mg urea per 100 kcal. More preferably the nutritional composition comprises 30 to 90 mg per 100 kcal, even more preferably 30 to 75 mg per 100 kcal, most preferably 30 to 55 mg urea per 100 kcal. Preferably the amount of urea-nitrogen is 3.2 to 16% of the total nitrogen. Preferably the amount of urea-nitrogen is 20 to 100 wt %, more preferably 20 to 80 wt % of total non-protein nitrogen.

When referring to amounts to 100 ml this relates to packed ready-to-drink liquid products, but also to products that are ready to drink after they have been reconstituted with water from a powder or a concentrate according to instructions.

### Protein Levels

The nutritional composition according to the present invention preferably comprises protein. The level of total protein is derived from the commonly known Kjeldahl nitrogen analysis and multiplication by a factor of 6.25. This gives a level of total protein, sometimes also referred to as crude protein level. Hence total protein is the sum of protein equivalent as defined herein plus non-protein nitrogen (NPN). Methods to determine protein and peptides and free amino acids are known in the art. For example a method wherein first protein an peptides are precipitated followed by common protein determination and free amino acids can be determined in the non-precipitated fraction by HPLC with pre-column derivatization with fluorenylmethyl-chloroformate and UV and fluorescence detection is suitable.

In case protein equivalent is present, the total protein in the present nutritional composition preferably provides 6.4 to 15% of the total calories. Preferably the nutritional composition comprises total protein that provides 7.2 to 11% of the total calories. More preferably the present nutritional composition comprises 1.6 to 3.5 g total protein per 100 kcal, more preferably 1.8 to 2.5 g per 100 kcal, even more preferably 1.8 to 2.4 g per 100 kcal and even more preferably 1.8 to 2.1 g per 100 kcal, most preferably from 1.85 to 2.0 g total protein per 100 kcal. Preferably the composition comprises 2.1 g or less total protein per 100 kcal. A low total protein concentration advantageously is closer to human milk as human milk comprises a lower amount of protein based on total calories than cow's milk. Based on dry weight the present nutritional composition preferably comprises 7.8 to 17 wt % total protein, more preferably 8.7 to 12.2 wt %, even more preferably 9.2 to 11.7 wt % total protein based on dry weight. Based on a ready-to-drink liquid product the nutritional composition preferably comprises 1.1 to 2.4 g total protein per 100 ml, more preferably 1.2 to 1.7 g, even more preferably between 1.3 and 1.6 g total protein protein per 100 ml.

If present, the protein equivalent in the present nutritional composition preferably provides 6 to 14.5% of the total calories. Preferably the nutritional composition comprises protein equivalent that provides 6.8 to 10% of the total calories. More preferably the present nutritional composition comprises 1.5 to 3.45 g protein equivalent per 100 kcal, more preferably 1.7 to 2.5 g per 100 kcal, even more preferably 1.8 to 2.4 g per 100 kcal and even more preferably 1.8 to 2.0 g protein equivalent per 100 kcal. Based on dry weight the present nutritional composition preferably comprises 7.3 to 16.5 wt % protein equivalent, more preferably 8.3 to 12.1 wt %, even more preferably 9.0 to 11.6 wt % protein equivalent based on dry weight. Based on a ready-to-drink liquid product the nutritional composition preferably comprises 1.0 to 2.35 g protein equivalent per 100 ml, more preferably 1.1 to 1.65 g, even more preferably between 1.25 and 1.6 g protein equivalent per 100 ml.

When present, the source of the protein should be selected in such a way that the minimum requirements for essential amino acid content are met and satisfactory growth is ensured. Hence protein sources based on cows' milk proteins such as whey, casein and mixtures thereof and proteins based on soy, potato or pea are preferred. Also protein from the milk of goats or sheep is suitable to provide the minimum requirements for essential amino acid content so that satisfactory growth is ensured.

Hence protein sources based on cows' milk proteins, goat milk protein or sheep milk protein are preferred, such as whey, casein and mixtures thereof. In case whey proteins are used, the protein source is preferably based on acid whey or sweet whey, whey protein isolate or mixtures thereof.

Preferably the nutritional composition comprises at least 3 wt % casein based on dry weight. Preferably the casein is intact and/or non-hydrolysed. Preferably, the nutritional composition comprises at least 90 wt % cow's milk proteins based on total protein. In one embodiment, preferably, the nutritional composition comprises at least 90 wt % sheep milk proteins based on total protein. In one embodiment, preferably, the nutritional composition comprises at least 90 wt % goat milk proteins based on total protein.

### Human Milk Oligosaccharides (HMOs)

The nutritional composition of the present invention comprises one or more human milk oligosaccharide selected from the group consisting of 2′-FL, 3-FL, 3′-SL and 6′-SL. Human milk oligosaccharides are present in human milk and are non-digestible oligosaccharides. Preferably the nutritional composition according to the invention comprises at least 0.005 g/100 ml of the one or more human milk oligosaccharide(s).

Preferably, a nutritional composition according to the invention comprises 0.005 g to 1.5 g human milk oligosaccharides (HMOs) per 100 ml, preferably 0.01 g to 1.5 g HMOs per 100 ml, more preferably 0.02 g to 0.75 g, even more preferably 0.04 g to 0.3 g HMOs per 100 ml. Based on dry weight, the present nutritional composition preferably comprises 0.038 wt % to 12 wt % HMOs, preferably 0.075 wt % to 12 wt % HMOs, more preferably 0.15 wt % to 6 wt % HMOs, even more preferably 0.3 wt % to 2.5 wt % HMOs.

Based on energy, the present nutritional composition preferably comprises 0.008 g to 2.5 g HMOs per 100 kcal, preferably 0.015 g to 2.5 g HMOs per 100 kcal, more preferably 0.03 g to 1.0 g HMOs per 100 kcal, even more preferably 0.06 g to 0.5 g HMOs per 100 kcal. A lower amount of HMOs will be less effective in stimulating growth of the bifidobacteria, whereas a too high amount will result in an increase the risk of osmotic diarrhea. This will counteract the beneficial effects of the mix on the intestinal health.

Preferably, a nutritional composition according to the invention comprises at least 0.005 g of the sum of 2′-FL, 3-FL, 3′-SL and 6′-SL per 100 ml, more preferably at least 0.01 g, more preferably at least 0.02 g, even more preferably at least 0.04 g of the sum of 2′-FL, 3-FL, 3′-SL and 6′-SL per 100 ml. Based on dry weight, the present nutritional composition preferably comprises at least 0.038 wt % of the sum of 2′-FL, 3-FL, 3′-SL and 6′-SL, more preferably at least 0.075 wt %, more preferably at least 0.15 wt % of the sum of 2′-FL, 3-FL, 3′-SL and 6′-SL, even more preferably at least 0.3 wt %. Based on energy, the present nutritional composition preferably comprises at least 0.008 g of the sum of 2′-FL, 3-FL, 3′-SL and 6′-SL per 100 kcal, more preferably at least 0.015 g per 100 kcal, more preferably at least 0.03 g per 100 kcal, even more preferably at least 0.06 g per 100 kcal.

Preferably, a nutritional composition according to the invention comprises 0.005 g to 1.5 g of the sum of 2′-FL, 3-FL, 3′-SL and 6′-SL per 100 ml, preferably 0.01 g to 1.5 g, more preferably 0.02 g to 0.75 g, even more preferably 0.04 g to 0.3 g of the sum of 2′-FL, 3-FL, 3′-SL and 6′-SL per 100 ml. Based on dry weight, the present nutritional composition preferably comprises 0.038 wt % to 12 wt % of the sum of 2′-FL, 3-FL, 3′-SL and 6′-SL, preferably 0.075 wt % to 12 wt %, more preferably 0.15 wt % to 6 wt % of the sum of 2′-FL, 3-FL, 3′-SL and 6′-SL, even more preferably 0.3 wt % to 2.5 wt %. Based on energy, the present nutritional composition preferably comprises 0.008 g to 2.5 g of the sum of 2′-FL, 3-FL, 3′-SL and 6′-SL per 100 kcal, preferably 0.015 g to 2.5 g of the sum of 2′-FL, 3-FL, 3′-SL and 6′-SL per 100 kcal, more preferably 0.03 g to 1.0 g per 100 kcal, even more preferably 0.06 g to 0.5 g per 100 kcal. 2′-Fucosyllactose (2′-FL) is an oligosaccharide present in human milk (HMO). It is not present in bovine milk. It consists of three monose units, fucose, galactose and glucose linked together. Lactose is a galactose unit linked to a glucose unit via a beta1,4 linkage. A fucose unit is linked to a galactose unit of a lactose molecule via an alpha1,2 linkage. 2′-FL is commercially available, for instance from Sigma-Aldrich or Christian Hansen-Jennewein. 2′-FL is split by extracellular enzymes of B. bifidum into lactose and fucose. B. longum ssp infantis will internalize 2′-FL and metabolize it to fucose and lactose. Fucose and lactose will be consumed.

Preferably the nutritional composition according to the invention comprises 2′-FL. Preferably the nutritional composition according to the invention comprises as a HMOs essentially 2′-FL, that means at least 95 wt % of the HMOs consists of 2′-FL. Preferably, a nutritional composition according to the invention comprises at least 0.005 g 2′-FL per 100 ml, more preferably at least 0.01 g, more preferably at least 0.02 g, even more preferably at least 0.04 g 2′-FL per 100 ml. Based on dry weight, the present nutritional composition preferably comprises at least 0.038 wt % 2′-FL, more preferably at least 0.075 wt %, more preferably at least 0.15 wt %, even more preferably at least 0.3 wt % 2′-FL. Based on energy, the present nutritional composition preferably comprises at least 0.008 g 2′-FL per 100 kcal, more preferably at least 0.015 g, more preferably at least 0.03 g 2′-FL per 100 kcal, even more preferably at least 0.06 g 2′-FL per 100 kcal.

Preferably, a nutritional composition according to the invention comprises 0.01 g to 1 g 2′-FL per 100 ml, more preferably 0.02 g to 0.5 g, even more preferably 0.04 g to 0.2 g 2′-FL per 100 ml. Based on dry weight, the present nutritional composition preferably comprises 0.075 wt % to 7.5 wt % 2′-FL, more preferably 0.15 wt % to 3.8 wt % 2′-FL, even more preferably 0.3 wt % to 1.5 wt % 2′-FL. Based on energy, the present nutritional composition preferably comprises 0.015 g to 1.5 g 2′-FL per 100 kcal, more preferably 0.03 g to 0.75 g 2′-FL per 100 kcal, even more preferably 0.06 g to 0.3 g 2′-FL per 100 kcal. A lower amount of 2′-FL will be less effective in stimulating growth of the bifidobacteria, whereas a too high amount may increase the risk of osmotic diarrhea. This will counteract the beneficial effects of the mix on the intestinal health. 3-Fucosyllactose (3-FL) is an oligosaccharide present in human milk (HMO). It is not present in bovine milk. It consists of three monose units, fucose, galactose and glucose linked together. Lactose is a galactose unit linked to a glucose unit via a beta1,4 linkage. A fucose unit is linked to a galactose unit of a lactose molecule via or via an alpha-1,3 linkage to the glucose unit of a lactose. 3-FL is commercially available, for instance from Sigma-Aldrich or Christian Hansen-Jennewein. 3-FL is split by extracellular enzymes of B. bifidum into lactose and fucose. B. longum ssp infantis will internalize 3-FL and metabolize it to fucose and lactose. Fucose and lactose will be consumed.

Preferably, a nutritional composition according to the invention comprises at least 0.005 g 3-FL per 100 ml, more preferably at least 0.01 g, even more preferably at least 0.02 g 3-FL per 100 ml. Based on dry weight, the present nutritional composition preferably comprises at least 0.04 wt % 3-FL, more preferably at least 0.075 wt % 3-FL, even more preferably at least 0.15 wt % 3-FL. Based on energy, the present nutritional composition preferably comprises at least 0.008 g 3-FL per 100 kcal, more preferably at least 0.015 g 3-FL per 100 kcal, even more preferably at least 0.03 g 3-FL per 100 kcal.

Preferably, a nutritional composition according to the invention comprises 0.005 g to 0.5 g 3-FL per 100 ml, more preferably 0.01 g to 0.25 g, even more preferably 0.02 g to 0.10 g 3-FL per 100 ml. Based on dry weight, the present nutritional composition preferably comprises 0.04 wt % to 4 wt % 3-FL, more preferably 0.075 wt % to 2.0 wt % 3-FL, even more preferably 0.15 wt % to 0.75 wt % 3-FL. Based on energy, the present nutritional composition preferably comprises 0.008 g to 0.75 g 3-FL per 100 kcal, more preferably 0.015 g to 0.04 g 3-FL per 100 kcal, even more preferably 0.03 g to 0.2 g 3-FL per 100 kcal. A lower amount of 3-FL will be less effective in stimulating growth of the bifidobacteria, whereas a too high amount may increase the risk of osmotic diarrhea. This will counteract the beneficial effects of the mix on the intestinal health. 3′-SL is an acidic HMO present in human milk. It consists of three monose units, sialic acid, galactose and glucose linked together. Lactose is a galactose unit linked to a glucose unit via a beta1,4 linkage. A sialic acid unit is linked to a galactose unit of a lactose molecule via an alpha 2,3 linkage. 3′-SL is commercially available, for instance from Sigma-Aldrich or Christian Hansen-Jennewein. 3′-Sialyllactose (3′-SL) is split by extracellular enzymes of B. bifidum into lactose and sialic acid. The sialic acid is not consumed by B. bifidum. B. longum ssp infantis will internalize 3′-SL and metabolize it to lactose and sialic acid. The sialic acid and lactose can be consumed.

Preferably, a nutritional composition according to the invention comprises at least 0.005 g 3′-SL per 100 ml, more preferably at least 0.01 g, even more preferably at least 0.02 g 3′-SL per 100 ml. Based on dry weight, the present nutritional composition preferably comprises at least 0.04% 3′-SL, more preferably at least 0.075 wt %, even more preferably at least 0.15 wt % 3′-SL. Based on energy, the present nutritional composition preferably comprises at least 0.008 g 3′-SL per 100 kcal, more preferably at least 0.015 g 3′-SL per 100 kcal, even more preferably at least 0.03 g 3′-SL per 100 kcal.

Preferably, a nutritional composition according to the invention comprises 0.005 g to 0.5 g 3′-SL per 100 ml, more preferably 0.01 g to 0.25 g, even more preferably 0.02 g to 0.1 g 3′-SL per 100 ml. Based on dry weight, the present nutritional composition preferably comprises 0.04 wt % to 4 wt % 3′-SL, more preferably 0.075 wt % to 2.0 wt % 3′-SL, even more preferably 0.15 wt % to 0.75 wt % 3′-SL. Based on energy, the present nutritional composition preferably comprises 0.008 g to 0.75 g 3′-SL per 100 kcal, more preferably 0.015 g to 0.04 g 3′-SL per 100 kcal, even more preferably 0.03 g to 0.2 g 3′-SL per 100 kcal. A lower amount of 3′-SL will be less effective in stimulating growth of the bifidobacteria, whereas a too high amount may also increase the risk of osmotic diarrhea. This will counteract the beneficial effects of the mix on the intestinal health. 6′-SL is an acidic HMO present in human milk. It consists of three monose units, sialic acid, galactose and glucose linked together. Lactose is a galactose unit linked to a glucose unit via a beta1,4 linkage.

A sialic acid unit is linked to a galactose unit of a lactose molecule via an alpha 2,6 linkage. 6′-SL is commercially available, for instance from Sigma-Aldrich or Christian Hansen-Jennewein. 6′-Sialyllactose (6-′SL) is split by extracellular enzymes of B. bifidum into lactose and sialic acid. Sialic acid is not consumed. B. longum ssp infantis will internalize 6′-SL and metabolize it into lactose and sialic acid. The sialic acid and lactose will be consumed.

Preferably, a nutritional composition according to the invention comprises at least 0.005 g 6′-SL per 100 ml, more preferably at least 0.01 g, even more preferably at least 0.02 g 6′-SL per 100 ml. Based on dry weight, the present nutritional composition preferably comprises at least 0.04 wt % 6′-SL, more preferably at least 0.075 wt % 6′-SL, even more preferably at least 0.15 wt % 6′-SL. Based on energy, the present nutritional composition preferably comprises at least 0.008 g 6′-SL per 100 kcal, more preferably at least 0.015 g 6′-SL per 100 kcal, even more preferably at least 0.3 g 6′-SL per 100 kcal.

Preferably, a nutritional composition according to the invention comprises 0.005 g to 0.5 g 6′-SL per 100 ml, more preferably 0.01 g to 0.25 g, even more preferably 0.02 mg to 0.1 g 6′-SL per 100 ml. Based on dry weight, the present nutritional composition preferably comprises 0.04 wt % to 4 wt % 6′-SL, more preferably 0.075 wt % to 2.0 wt % 6′-SL, even more preferably 0.15 wt % to 0.75 wt % 6′-SL. Based on energy, the present nutritional composition preferably comprises 0.008 g to 0.75 g 6′-SL per 100 kcal, more preferably 0.015 g to 0.04 g 6′-SL per 100 kcal, even more preferably 0.03 g to 0.15 g 6′-SL per 100 kcal. A lower amount of 6′-SL will be less effective in stimulating growth of the bifidobacteria, whereas a too high amount may also increase the risk of osmotic diarrhea. This will counteract the beneficial effects of the mix on the intestinal health.

Most preferably the nutritional composition comprises 2′-FL. 2′-FL was demonstrated to be very effective in the specific synbiotic mix, and it is also the most abundant milk oligosaccharide in human milk.

Not all HMOs will have the same effect as the four specific HMOs selected above. Lacto-N-tetraose (LNT) and lacto-N-neotetraose (LNnT) are abundant HMOs in human milk and many bifidobacteria strains are able to take up and metabolize these HMOs, so no surprising effect in combination with the presently required bifidobacteria strains is expected. However, these HMOs may be present in addition to the HMOs of the present invention.

The nutritional composition of the present invention comprises at least one of 2′-FL, 3-FL, 3′-SL and 6′-SL, preferably at least 2′-FL. Preferably the nutritional composition comprises mixtures of HMOs, preferably a mixture of 2′-FL, 3-FL, 3′-SL, and 6′-SL, more preferably a mixture of 2′-FL, 3-FL, 3′-SL, 6′-SL and LNT.

Alternatively, the nutritional composition of the present invention comprises at least four, preferably at least five human milk oligosaccharides selected from the group consisting of 2′-fucosyllactose, 3-fucosyllactose, 3′-sialyllactose, 6′-sialyllactose, lacto-N-tetraose, lactol-N-neotetraose and difucosyllactose.

### Beta-Galacto-Oligosaccharides

Preferably beta-galacto-oligosaccharides (bGOS) are present in the nutritional composition, preferably bGOS with a DP of 4 or more. Preferably the bGOS has predominantly beta1,4 linkages. bGOS is a non-digestible oligosaccharide (NDO). Thus in one embodiment, the nutritional composition according to the invention further comprises non-digestible galacto-oligosaccharide comprising beta1,4 linkages, in particular beta1,4 linkages between the galactose units, and having a degree of polymerization of at least 4.

A suitable way to form bGOS is to treat lactose with beta-galactosidases. Dependent on the specificity of the enzyme used, a galactose unit is hydrolysed from lactose and coupled to another lactose unit via a beta-linkage to form a trisaccharide. A galactose unit may also be coupled to another single galactose unit to form a disaccharide. Subsequent galactose units are coupled to form oligosaccharides. A suitable way to prepare beta1,4 GOS is by using the beta-galactosidase from Bacillus circulans or Cryptococcus laurentii. A commercially available source of bGOS is Vivinal® GOS from FrieslandCampina Domo (Amersfoort, The Netherlands). Vivinal® GOS comprises bGOS mainly with DP2-8 and with beta1,4 linkages being more predominant. Van Leeuwen et al, 2014, Carbohydrate Res 400: 59-73 disclose that commercial Vivinal® GOS has about 1.5% Gal, 18.5% Glu, 42.5% DP2 (including the 21% lactose), 23.6% bGOS with DP3, 10.2% bGOS DP4, 3.0% bGOS DP5 and <0.5% bGOS DP6 and higher. Based on bGOS, so excluding lactose, galactose, and glucose, the amount of bGOS with DP4 or higher in Vivinal® GOS is thus about 22.4% based on total bGOS. Other suitable sources of bGOS with beta1,4 linkages and comprising structures with DP4 or higher are Cup Oligo from Nissin Sugar, and galactan coming from potato tuber pectin (Megazyme Int). It is noted that in the context of the present invention oligosaccharides like beta1,3′-galactosyllactose, beta1,4′-galactosyllactose, beta1,6′-galactosyllactose may be present as part of the bGOS fraction and thus are not considered to be human milk oligosaccharides.

Preferably the nutritional composition comprises at least 0.25 g bGOS per 100 ml, more preferably at least 0.4 g even more preferably at least 0.6 g per 100 ml. Preferably the nutritional composition does not comprise more than 2.5 g bGOS per 100 ml, preferably not more than 1.5 g, more preferably not more than 1.0 g per 100 ml. More preferably, the nutritional composition according to the present invention comprises bGOS in an amount of 0.25 to 2.5 g/100 ml, even more preferably in an amount of 0.4 to 1.5 g/100 ml, even more preferably in an amount of 0.6 to 1.0 g/100 ml.

Preferably the nutritional composition comprises at least 1.75 wt % bGOS based on dry weight of the total composition, more preferably at least 2.8 wt %, even more preferably at least 4.2 wt % based on dry weight of the total composition. Preferably the nutritional composition does not comprise more than 17.5 wt % bGOS based on dry weight of the total composition, more preferably not more than 10.5 wt %, even more preferably not more than 7 wt % based on dry weight of the total composition. The nutritional composition according to the present invention preferably comprises bGOS in an amount of 1.75 to 17.5 wt %, more preferably in an amount of 2.8 to 10.5 wt %, most preferably in an amount of 4.2 to 7 wt %, all based on dry weight of the total composition. Preferably the nutritional composition according to the present invention comprises at least 0.35 g bGOS per 100 kcal, more preferably at least 0.6 g, even more preferably at least 0.8 g per 100 kcal. Preferably the nutritional composition does not comprise more than 3.7 g bGOS per 100 kcal, preferably not more than 2.5 g per 100 kcal, more preferably not more than 1.5 g per 100 kcal. More preferably, the nutritional composition according to the present invention comprises bGOS in an amount of 0.35 to 3.7 g per 100 kcal, even more preferably in an amount of 0.6 to 2.5 g per 100 ml, even more preferably in an amount of 0.8 to 1.5 g per 100 ml. Lower amounts result in a less effective composition, whereas the presence of higher amounts of bGOS may result in side-effects such as osmotic disturbances, abdominal pain, bloating, gas formation and/or flatulence.

In one embodiment, the nutritional composition according to the invention preferably comprises galacto-oligosaccharide comprising beta1,4 linkages having a DP of at least 4 in an amount of at least 0.05 g/100 ml. Preferably the nutritional composition comprises at least 0.08 g bGOS having a DP of at least 4 per 100 ml, even more preferably at least 0.12 g per 100 ml. Preferably the nutritional composition does not comprise more than 0.5 g bGOS having a DP of at least 4 per 100 ml, preferably not more than 0.3 g, more preferably not more than 0.2 g. More preferably, the nutritional composition according to the present invention comprises bGOS having a DP of at least 4 in an amount of 0.05 to 0.5 g/100 ml, even more preferably in an amount of 0.08 to 0.3 g/100 ml, even more preferably in an amount of 0.12 to 0.2 g/100 ml.

Preferably the nutritional composition comprises at least 0.35 wt % bGOS with DP4 or higher based on dry weight of the total composition, more preferably at least 0.6 wt %, even more preferably at least 0.8 wt %, all based on dry weight of the total composition. Preferably the nutritional composition does not comprise more than 3.5 wt % bGOS with DP4 or higher based on dry weight of the total composition, more preferably not more than 2 wt %, even more preferably not more than 1.5 wt %. The nutritional composition according to the present invention preferably comprises bGOS with DP4 or higher in an amount of 0.35 to 3.5 wt %, more preferably in an amount of 0.6 to 2 wt %, most preferably in an amount of 0.8 to 1.5 wt %, all based on dry weight of the total composition.

Preferably the nutritional composition according to the present invention comprises at least 0.07 g bGOS with DP4 or higher per 100 kcal, more preferably at least 0.12 g, even more preferably at least 0.16 g per 100 kcal. Preferably the nutritional composition does not comprise more than 0.75 g bGOS with DP4 or higher per 100 kcal, preferably not more than 0.5 g per 100 kcal, more preferably not more than 0.3 g per 100 kcal. More preferably, the nutritional composition according to the present invention comprises bGOS with DP4 or higher in an amount of 0.07 to 0.75 g per 100 kcal, even more preferably in an amount of 0.12 to 0.5 g per 100 ml, even more preferably in an amount of 0.16 to 0.3 g per 100 ml. Lower amounts result in a less effective composition, whereas the presence of higher amounts of bGOS may result in side-effects such as osmotic disturbances, abdominal pain, bloating, gas formation and/or flatulence.

Preferably the nutritional composition according to the present invention also comprises fructo-oligosaccharides (FOS). Preferably the fructo-oligosaccharides have a DP or average DP in the range of 2 to 250, more preferably 2 to 100, even more preferably 10 to 60. Preferably the nutritional composition comprises long chain fructo-oligosaccharides (IcFOS), also referred to as non-digestible polyfructose, with an average DP of at least 11, more preferably at least 20. FOS suitable for use in the composition of the invention is also readily commercially available, e.g. RaftilineHP® (Orafti). Preferably the nutritional composition according to the present invention comprises at least 25 mg FOS, preferably polyfructose with an average DP of at least 11, more preferably of at least 20, per 100 ml, more preferably at least 40 mg even more preferably at least 60 mg per 100 ml. Preferably the composition does not comprise more than 250 mg FOS, preferably polyfructose with an average DP of at least 11, more preferably of at least 20, per 100 ml, more preferably not more than 150 mg per 100 ml and most preferably not more than 100 mg per 100 ml. The amount of FOS, preferably polyfructose with an average DP of at least 11, more preferably of at least 20, is preferably 25 to 250 mg per 100 ml, preferably 40 to 150 g per 100 ml, more preferably 60 to 100 g per 100 ml. Preferably the nutritional composition according to the present invention comprises at least 0.15 wt % FOS, preferably polyfructose with an average DP of at least 11, more preferably of at least 20, based on dry weight, more preferably at least 0.25 wt %, even more preferably at least 0.4 wt %. Preferably the composition does not comprise more than 1.5 wt % FOS, preferably polyfructose with an average DP of at least 11, more preferably of at least 20, based on dry weight of the total composition, more preferably not more than 2 wt %. The presence of FOS, preferably polyfructose with an average DP of at least 11, more preferably of at least 20, together with bGOS shows a further improved effect on the microbiota and its SCFA production, which aid the restoration to a balanced microbiota after a disturbing event.

Preferably the present nutritional composition comprises a mixture of bGOS and FOS. Preferably the mixture of bGOS and FOS is present in a weight ratio of from 1/99 to 99/1, more preferably from 1/19 to 19/1, more preferably from 1/1 to 19/1, more preferably from 2/1 to 15/1, more preferably from 5/1 to 12/1, even more preferably from 8/1 to 10/1, even more preferably in a ratio of about 9/1. This weight ratio is particularly advantageous when the bGOS have a low average DP and FOS has a relatively high DP. Most preferred is a mixture of bGOS with an average DP below 10, preferably below 6, and FOS with an average DP above 7, preferably above 11, even more preferably above 20. Preferably FOS is polyfructose with an average DP of at least 11, more preferably with an average DP of at least 20.

Preferably the weight ratio of the sum of 2′-FL, 3-FL, 3′-SL and 6′-SL to bGOS is 1:1 to 1:40, more preferably 1:1.5 to 1:20.

Preferably the weight ratio of 2′-FL to bGOS is 1:1 to 1:40, more preferably 1:1.5 to 1:20.

Preferably the weight ratio of the sum of 2′-FL, 3-FL, 3′-SL and 6′-SL to bGOS to IcFOS is 1:(1-40) (0.1-4), more preferably 1:(1.5-20):(0.15-2.0).

Preferably the weight ratio of 2′-FL to bGOS to IcFOS is 1:(1-40):(0.1-4), more preferably 1:(1.5-20):(0.15-2.0). These ratios will improve a cross-feeding interaction (syntrophic effect) of the mix of specific bifidobacteria and non-digestible oligosaccharides.

Preferably the weight ratio of the sum of 2′-FL, 3-FL, 3′-SL and 6′-SL to bGOS with DP4 or more is 1:0.25 to 1:10 more preferably 1:0.3 to 1:5.

Preferably the weight ratio of 2′-FL to bGOS with DP4 or more is 1:0.25 to 1:10, more preferably 1:0.3 to 1:5.

Preferably the weight ratio of the sum of 2′-FL, 3-FL, 3′-SL and 6′-SL to bGOS with DP4 or more to IcFOS is 1:(0.25-10):(0.1-4), more preferably 1:(0.3-5):(0.15-2.0).

Preferably the weight ratio of 2′-FL to bGOS with DP4 or more to IcFOS is 1:(0.25-10):(0.1-4), more preferably 1:(0.3-5):(0.15-2.0).

These ratios will improve a cross-feeding interaction (syntrophic effect) of the mix of specific Bifidobacterium species and non-digestible oligosaccharides.

### Bifidobacterium Mix

The present nutritional composition comprises at least two strains of bifidobacteria, of which one is a Bifidobacterium bifidum that is able to express at least one extracellular enzyme selected from a fucosidase and a sialidase and one is a Bifidobacterium longum ssp infantis able to express urease. Preferably a third strain is present, which is a strain of Bifidobacterium breve that is not able to express urease but that is able to metabolize a saccharide selected from L-fucose and sialic acid and able to express extracellular beta1,4 endogalactanase.

The present nutritional composition preferably contains at least 2.10³ colony forming units (cfu) bifidobacteria per gram dry weight of the nutritional composition, more preferably at least 2.10⁴ cfu, even more preferably at least 2.10⁵ cfu bifidobacteria per gram dry weight of the nutritional composition. The present nutritional composition preferably contains 2.10³ to 2.10¹³ colony forming units (cfu) bifidobacteria per gram dry weight of the nutritional composition, preferably 2.10⁴ to 2.10¹², more preferably 2.10⁵ to 2.10¹⁰, most preferably 2.10⁵ to 2.10⁹ cfu bifidobacteria per gram dry weight of the nutritional composition.

Preferably each Bifidobacterium strain of the mix according to the invention is able to hydrolyze and metabolize lactose by lactase or beta1,4-galactosidase. This enables to metabolize the lactose that is a result of the degradation of the human milk oligosaccharide and of beta-galacto-oligosaccharides (bGOS) that are optionally present.

Bifidobacterium longum subsp. infantis

The nutritional composition comprises a strain of Bifidobacterium longum subsp. infantis (B. infantis).

Bifidobacterium longum subsp. infantis is a Gram-positive, anaerobic, branched rod-shaped bacterium. The present B. infantis preferably has at least 95% identity of the 16 S rRNA sequence when compared to the type strain of B. longum subsp. infantis ATCC 15607, more preferably at least 97% identity (Stackebrandt & Goebel, 1994, Int. J. Syst. Bacteriol. 44:846-849). The strain of Bifidobacterium longum subspecies infantis is able to internalize a human milk oligosaccharide selected from the group consisting of 2′-fucosyllactose, 3-fucosyllactose, 3′-sialyllactose, and 6′-sialyllactose. The B. infantis strain is able to use urea as nitrogen source, as it is able to express urease activity. Urease is an enzyme that splits urea into CO₂ and ammonia. The urease positive B. infantis will release CO₂ and ammonia from the urea and will thus support the growth of urease negative bifidobacteria, by providing ammonia as N source and CO₂ which is an important factor for good bifidobacterial growth.

Preferred B. infantis strains to be optionally included in the nutritional composition according to the present invention are those isolated from the faeces of healthy human milk-fed infants. Typically, these are commercially available from producers of lactic acid producing bacteria, but they can also be directly isolated from faeces, identified, characterised and produced. B. infantis strains are available.

Suitable and available B. infantis strains are B. infantis M-63 (LMG 23728, Morinaga), BB-02 (Christian Hansen, DSM 33361), or R0033 (Lallemand). These strains are urease positive. Whether a B. infantis strain is urease positive can be easily be determined by methods known in the art.

The present nutritional composition preferably contains at least 10³ cfu B. infantis per gram dry weight of the nutritional composition, more preferably at least 10⁴ cfu even more preferably at least 10⁵ cfu B. infantis per gram dry weight of the nutritional composition. If present, the present nutritional composition preferably contains 10³ to 10¹³ colony forming units (cfu) B. infantis per gram dry weight of the present composition, preferably 10⁴ to 10¹² cfu more preferably 10⁵ to 10¹⁰ cfu most preferably from 10⁵ to 10⁹ cfu B. infantis per gram dry weight of the nutritional composition.

Bifidobacterium bifidum The nutritional composition comprises a strain of Bifidobacterium bifidum. This species is key to the mix and is able to express extracellularly enzymes that degrade human milk oligosaccharides. In particular the B. bifidum is able to express at least one of a fucosidase and a sialidase and this ability enables the hydrolysis of one or more of the human milk oligosaccharides (HMO) 2′-fucosyllactose (2′-FL), 3-fucosyllactose (3-FL), 3′-sialyllactose (3′-SL), and 6′-sialyllactose (6′-SL).

It was found that surprisingly a mixture of B. infantis, B, bifidum, and one or more HMOs selected from 2′-FL, 3-FL, 3′-SL and 6′-SL, and urea resulted in a synergistic growth of bifidobacteria and organic acid formation.

In one embodiment the Bifidobacterium bifidum in the present nutritional composition is able to grow on 2′-FL and hydrolyses it extracellularly to fucose and lactose. In one embodiment the Bifidobacterium bifidum in the present nutritional composition is able to grow on 3-FL and hydrolyses it extracellularly to fucose and lactose. In one embodiment the Bifidobacterium bifidum in the present nutritional composition is able to grow on 3′-SL and hydrolyses it extracellularly to sialic acid and lactose. In one embodiment the Bifidobacterium bifidum in the present nutritional composition is able to grow on 6′ SL and hydrolyses it extracellularly to sialic acid and lactose. Lactose is taken up by a transport system and split internally by a lactase into glucose and galactose. Bifidobacterium bifidum is not able to take up and metabolize fucose and sialic acid. These degradation products are therefore available for other bacteria to use as carbon and energy source. B. bifidum is not able to use urea as nitrogen source and is dependent on B. infantis if urea is the sole nitrogen source.

Bifidobacterium bifidum is a Gram-positive, anaerobic, branched rod-shaped bacterium. The B. bifidum according to the present invention preferably has at least 95% identity of the 16 S rRNA sequence when compared to the type strain of B. bifidum ATCC 29521, more preferably at least 97% identity (Stackebrandt & Goebel, 1994, Int. J. Syst. Bacteriol. 44:846-849). Preferred B. bifidum strains are those isolated from the faeces of healthy human milk-fed infants. Typically, these are commercially available from producers of lactic acid producing bacteria, but they can also be directly isolated from faeces, identified, characterised, and produced. Examples of suitable and available B. bifidum strains are B. bifidum R0071 from Lallemand or B. bifidum Bb-06 (Dupont Dansico). Most preferably, the B. bifidum is B. bifidum CNCM I-4319. This strain was deposited under Budapest treaty at the Collection National de Cultures de Microorganisms (CNCM) at Institut Pasteur, 25 Rue de Dr Roux, 75724 Paris by Compagnie Gervais Danone on 19 May 2010. B. bifidum CNCM I-4319 is a strain originally isolated from the infant microbiota of a healthy baby born in the Netherlands. This strain is especially preferred because it has the ability to protect the intestinal epithelial barrier measured by transepithelial electrical resistance (TEER) in an in vitro model (WO 2011/148358) and in an animal model it was shown to restore gut integrity and functionality from stress-induced and inflammatory damage (Tondereau at al., Microorganisms, 2020, 8, 1313). This is a characteristic that is especially beneficial under conditions when the intestinal microbiota is in disbalance. B. bifidum CNCM I-4319 has also been disclosed in U.S. Pat. No. 9,402,872.

The present nutritional composition preferably contains at least 10³ cfu B. bifidum per gram dry weight of the nutritional composition, more preferably at least 10⁴ cfu, even more preferably 10⁵ cfu B. bifidum per gram dry weight of the nutritional composition. The present nutritional composition preferably contains 10³ to 10¹³ colony forming units (cfu) B. bifidum per gram dry weight of the nutritional composition, preferably 10⁴ to 10¹² cfu, more preferably 10⁵ to 10¹⁰ cfu, most preferably 10⁵ to 10⁹ cfu B. bifidum per gram dry weight of the nutritional composition.

Bifidobacterium breve

Although a combination of B. infantis with B. breve alone shows an antagonistic effect on growth and fermentation when grown on the HMOs, in the presence of B. bifidum the antagonism has disappeared and there appears to be no negative effect on the synergistic interaction between B. bifidum and B. breve. This allows the option to include a selected B. breve strain that has known additional health effects.

Therefore, the nutritional composition preferably comprises a strain of Bifidobacterium breve. In particular a B. breve that is able to metabolize a saccharide selected from L-fucose and sialic acid. B. breve strains typically are able to metabolize fucose and sialic acid. Preferably the B. breve is able to express extracellular beta1,4 endogalactanase. This species is beneficial to the mix as it is able to take up and metabolize fucose and/or sialic acid produced by the B. bifidum, but is not able to directly grow on 2′-FL, 3-FL, 3′-SL, or 6′-SL. Thus in a preferred embodiment according to the invention the Bifidobacterium breve strain or strains is/are not able to express fucosidase and is/are not able to express sialidase, or in other words is/are not able to express an enzyme that hydrolyses 2′ fucosyllactose (2′-FL), 3-fucosyllactose (3-FL), 3′-sialyllactose (3′-SL) and 6′-sialyllactose (6′-SL). The preferred ability to express extracellular beta1,4 endogalactanase results in that the B. breve is able to release the galactose units from beta-galacto-oligosaccharides (bGOS) that have a degree of polymerization (DP) of 4 or above. An antagonistic effect was observed when B. breve was combined with B. infantis able to utilize human milk oligosaccharides such as 2′-FL, 3-FL, 3′-SL, or 6′-SL. However, in the additional presence of B. bifidum the antagonism was no longer observed. B. breve is urease negative and is dependent on B. infantis if urea is the sole nitrogen source.

Bifidobacterium breve is a Gram-positive, anaerobic, branched rod-shaped bacterium. The B. breve according to the present invention preferably has at least 95% identity of the 16 S rRNA sequence when compared to the type strain of B. breve ATCC 15700, more preferably at least 97% identity (Stackebrandt & Goebel, 1994, Int. J. Syst. Bacteriol. 44:846-849).

Preferred B. breve strains are those isolated from the faeces of healthy human milk-fed infants. Typically these are commercially available from producers of lactic acid producing bacteria, but they can also be directly isolated from faeces, identified, characterised and produced. Suitable B. breve strains are available.

B. breve strains can be divided into two groups. A group that expresses extracellular beta1,4 endogalactanase (encoded by the GalA gene) EC3.2.1.89, and strains that do not express this enzyme. B. breve strains that are able to express beta1,4 endogalactanase are able to grow well on bGOS comprising oligosaccharides with a DP of 4 or more, because they are able to utilize all the bGOS components, whereas B. breve strains that are beta1,4 endogalactanase negative grow to a lesser extent on bGOS as they are not able to degrade the bGOS with a high degree of polymerization. Beta1,4 endogalactanase is able to degrade galactan or potato derived pectic arabinogalactan into bGOS with a DP3. The galactotriose is subsequently transported into the cell and metabolized.

Whether a B. breve strain has the ability to express beta1,4 endogalactanase can be determined by a growth experiment on bGOS and analysis of the supernatant as described in O'Connel Motherway et al 2012 Microbial biotechnology, 6: 67-69.

Examples of suitable B. breve strains that express beta1,4 endogalactanase are B. breve UCC2003 (NCIMB 8807), C50, JCM7017, NCFB2258 and NCIMB8815. [JCM: Japan Collection of Microorganisms. LMG: BCCM/LMG Belgian Coordinated Collections of Microorganisms. NCFB, National collection of food bacteria, UK. NCIMB, National Collection of Industrial, Food and marine bacteria, UK]

Especially preferred is to use B. breve C50. B. breve C50 was deposited under deposit number CNCM I-2219, under the Budapest Treaty at the Collection Nationale de Cultures de Microorganism, at Institut Pasteur, 25 Rue du Dr Roux, Paris, France on 31 May 1999 by Compagnie Gervais Danone. This strain was published in WO 2001/001785 and in U.S. Pat. No. 7,410,653. This strain is known to have immune stimulating activity and is able to improve the microbiota by reducing pathogens like Clostridium, in particular Clostridium perfringens, and Bacteroides fragilis. Furthermore the strain can produce factors that downregulate intestinal inflammation (Heuvelin et al 2009, Plos one, 4: e5184). These are features that are especially beneficial under conditions when the intestinal microbiota is in disbalance. Another preferred Bifidobacterium breve to use is Bifidobacterium breve CNCM 1-5177. B. breve CNCM 1-5177 was deposited under the Budapest Treaty at the Collection Nationale de Cultures de Microorganism, at Institut Pasteur, 25 Rue du Dr Roux, Paris, France on 9 Mar. 2017 by Compagnie Gervais Danone.

The present nutritional composition preferably contains at least 10³ cfu B. breve per gram dry weight of the nutritional composition, more preferably at least 10⁴ cfu, even more preferably at least 10⁵ cfu B. breve per gram dry weight of the nutritional composition. The present nutritional composition preferably contains 10³ to 10¹³ colony forming units (cfu) B. breve per gram dry weight of the nutritional composition, preferably 10⁴ to 10¹² cfu, more preferably 10⁵ to 10¹⁰ cfu most preferably from 10⁵ to 10⁹ cfu B. breve per gram dry weight of the nutritional composition.

The present nutritional composition preferably contains at least 10³ cfu B. breve able to express beta1,4 endogalactanase per gram dry weight of the nutritional composition, more preferably at least 10⁴ cfu, even more preferably at least 10⁵ cfu B. breve able to express beta1,4 endogalactanase per gram dry weight of the nutritional composition. The present nutritional composition preferably contains 10³ to 10¹³ colony forming units (cfu) B. breve able to express beta1,4 endogalactanase per gram dry weight of the nutritional composition, preferably 10⁴ to 10¹² cfu, more preferably 10⁵ to 10¹⁰ cfu most preferably from 10⁵ to 10⁹ cfu B. breve able to express beta1,4 endogalactanase per gram dry weight of the nutritional composition.

In a preferred embodiment, the nutritional composition according to the invention comprises the human milk oligosaccharide 2′-fucosyllactose and the Bifidobacterium breve strain able to express beta1,4 endogalactanase, that is able to metabolize L-fucose.

For enablement of the present invention hereabove various bifidobacterial strains have been specifically identified. It is to be understood that the present invention is also enabled for bifidobacteria strains that are not identical to those specifically mentioned, but that have the same functional ability. In particular a natural variant or mutant of the specific Bifidobacterium longum ssp infantis mentioned above and that is capable of expressing urease, and in addition an intracellular fucosidase and/or sialidase is also encompassed by the present invention. Also, a natural variant or mutant of the specific Bifidobacterium bifidum mentioned above and that is capable of expressing at least one extracellular enzyme selected from a fucosidase and a sialidase, or in other words capable of expressing fucosidase and/or sialidase activity, is also encompassed by the present invention. Likewise, a natural variant or mutant of the specific Bifidobacterium breve mentioned above and that is capable to metabolize a saccharide selected from L-fucose and sialic acid is also encompassed by the present invention. Moreover, a natural variant or mutant of the specific Bifidobacterium breve mentioned above and that is capable to metabolize a saccharide selected from L-fucose and sialic acid and that is also capable of expressing extracellular beta1,4 endogalactanase, or in other words capable of expressing beta1,4 endogalactanase activity, is also encompassed by the present invention.

In the context of the present invention it is noted that ability to express extracellular fucosidase, sialidase or beta1,4 endogalactanase means that such enzyme activity is expressed extracellularly.

Preferably the nutritional composition of the present invention does not comprise B. longum subs. longum (B. longum). As the beneficial effect is very specific for the mix of B. bifidum, B. infantis and preferably a beta1,4 endogalactanase positive B. breve, addition of further species may disturb the cross-feeding interaction interaction.

Preferably the nutritional composition of the present invention does not contain lactobacilli, or in other words, does not comprise a Lactobacillus species. As the beneficial effect is very specific for the mix of B. bifidum and B. infantis and preferably a beta1,4 endogalactanase positive B. breve, addition of further species may disturb the cross-feeding interaction. Furthermore, lactobacilli are a relatively minor component early in life of a balanced microbiota compared to bifidobacteria. Preferably the nutritional composition of the present invention contains no more than three strains of bifidobacteria. Besides a strain from B. longum ssp infantis and B. bifidum, preferably as a third strain, a strain of B. breve is present. The cross-feeding interactions could be disturbed when further strains or different species are added.

The present nutritional composition preferably contains at least 10³ cfu B. bifidum and at least 10³ cfu B. infantis and if present, preferably 10³ cfu B. breve able to express beta1,4 endogalactanase, per gram dry weight of the nutritional composition, more preferably at least 10⁴ cfu of each of B. bifidum and B. infantis, and if present, preferably at least 10⁴ cfu B. breve able to express beta1,4 endogalactanase, even more preferably 10⁵ cfu of each of B. bifidum and B. infantis, and if present, preferably 10⁵ B. breve able to express beta1,4 endogalactanase, per gram dry weight of the nutritional composition.

The present nutritional composition preferably contains 10³ to 10¹³ colony forming units (cfu) B. bifidum and 10³ to 10¹³ colony forming units (cfu) B. infantis, and if present, preferably 10³ to 10¹³ colony forming units (cfu) B. breve able to express beta1,4 endogalactanase, per gram dry weight of the nutritional composition, preferably 10⁴ to 10¹² cfu of each of B. bifidum and B. infantis and if present, preferably 10⁴ to 10¹² cfu B. breve able to express beta1,4 endogalactanase, more preferably 10⁵ to 10¹⁰ cfu, most preferably 10⁵ to 10⁹ cfu of each of B. bifidum and B. infantis and if present, preferably 10⁵ to 10¹⁰ cfu, most preferably 10⁵ to 10⁹ cfu B. breve able to express beta1,4 endogalactanase, per gram dry weight of the nutritional composition.

Preferably the nutritional composition contains B. bifidum and B. infantis in a cfu ratio of 1:10³ to 10³:1, more preferably in a cfu ratio of 1:10² to 10²:1.

Preferably the nutritional composition contains B. bifidum, B. breve able to express extracellular beta1,4 endogalactanase and B. infantis in a cfu ratio of (1-10³):(1-10³):(1:10³), more preferably (1-10²):(1-10²):(1:10²), meaning that each strain may differ by a factor 10³, preferably a factor 10², in cfu from any other strain present.

As mentioned before, preferably each Bifidobacterium strain of the mix according to the invention is able to hydrolyze and metabolize lactose by lactase or beta1,4-galactosidase. This enables to metabolize the lactose that is a result of the degradation of the human milk oligosaccharide and of beta-galacto-oligosaccharides (bGOS) that are optionally present. Hence this concerns the B. bifidum, the B. breve, able to express beta1,4 endogalactanase, and the B. infantis.

### Nutritional Composition

In the context of the present invention, the term nutritional composition encompasses also a supplement to be added to nutrition. It refers to a synthetic or artificial or non-natural composition that is edible.

In an embodiment the nutritional composition according to the invention comprises digestible carbohydrate, lipid, protein, urea, non-digestible oligosaccharide, and lactic acid producing bacteria
wherein the urea is present in an amount of at least 1.13 wt %, preferably 1.13 to 5.6 wt %, based on total protein, at least 0.11 wt %, preferably 0.11 to 0.55 wt %, based on dry weight of the composition, and/or at least 15 mg, preferably 15 to 75 mg, per 100 ml,
wherein the non-digestible oligosaccharide is present in an amount of at least 1.5 wt %, preferably 1.5 to 15 wt %, based on dry weight of the composition, and/or at least 0.2 g, preferably 0.2 to 2 g, per 100 ml,
wherein the non-digestible oligosaccharide comprises beta-galacto-oligosaccharides and at least one human milk oligosaccharide selected from the group consisting of 2′-fucosyllacose, 3-fucosyllactose, 3′-sialyllactose and 6′-sialyllactose,
wherein the lactic acid producing bacteria comprise at least a urease positive Bifidobacterium longum ssp infantis, a urease negative Bifidobacterium bifidum able to able to express at least one extracellular enzyme selected from a fucosidase and a sialidase, and
wherein the nutritional composition is not mammalian milk.

The nutritional composition according to the invention preferably comprises digestible carbohydrate. The digestible carbohydrate preferably provides 30 to 80% of the total calories of the nutritional composition. Preferably the digestible carbohydrate provides 40 to 60% of the total calories. Based on calories the nutritional composition preferably comprises of 5 to 20 g of digestible carbohydrate per 100 kcal, more preferably 7.5 to 15 g. When in liquid form, e.g. as a ready-to-feed liquid, the nutritional composition preferably comprises 3.0 to 30 g digestible carbohydrate per 100 ml, more preferably 6.0 to 20, even more preferably 7.0 to 10.0 g per 100 ml. Based on dry weight the nutritional composition preferably comprises 20 to 80 wt %, more preferably 40 to 65 wt % digestible carbohydrate.

Preferred digestible carbohydrate sources are lactose, glucose, sucrose, fructose, galactose, maltose, starch and maltodextrin. Lactose is the main digestible carbohydrate present in human milk. Lactose advantageously has a low glycemic index. The nutritional composition preferably comprises lactose. The nutritional composition preferably comprises digestible carbohydrate, wherein at least 35 wt %, more preferably at least 50 wt %, more preferably at least 75 wt %, even more preferably at least 90 wt %, most preferably at least 95 wt % of the digestible carbohydrate is lactose. Based on dry weight the nutritional composition preferably comprises at least 25 wt % lactose, preferably at least 40 wt %.

The nutritional composition according to the present invention preferably comprises lipid. The lipid of the present nutritional composition preferably provides 3 to 7 g per 100 kcal of the nutritional composition, preferably the lipid provides 4 to 6 g per 100 kcal. When in liquid form, e.g. as a ready-to-feed liquid, the nutritional composition preferably comprises 2.1 to 6.5 g lipid per 100 ml, more preferably 3.0 to 4.0 g per 100 ml. Based on dry weight the present nutritional composition preferably comprises 12.5 to 40 wt % lipid, more preferably 19 to 30 wt %. Preferably the lipid comprises the essential fatty acids alpha-linolenic acid (ALA) and linoleic acid (LA). The lipid may be provided as free fatty acids, in triglyceride form, in diglyceride form, in monoglyceride form, in phospholipid form, or as a mixture of one of more of the above. Preferably the present nutritional composition contains at least one, preferably at least two lipid sources selected from the group consisting of rape seed oil (such as colza oil, low erucic acid rape seed oil and canola oil), high oleic sunflower oil, high oleic safflower oil, olive oil, marine oils, microbial oils, coconut oil, palm kernel oil and milk fat.

The nutritional composition according to the present invention preferably is intended for administration to infants and young children. The present nutritional composition is not human milk. The present nutritional composition is also not native cow's milk or native milk from another mammal. The terms “infant formula” or “follow on formula” or “young child formula” or “growing up milk” mean that it concerns a composition that is artificially made or in other words that it is synthetic. Hence in one embodiment the nutritional composition that is administered is an artificial infant formula or an artificial follow on formula or an artificial young child formula or an artificial growing up milk or a synthetic infant formula or a synthetic follow on formula or a synthetic young child formula or a synthetic growing up milk.

In the present context, infant formula refers to nutritional compositions, artificially made, intended for infants of 0 to about 4 to 6 months, preferably 6 months, of age and are intended as a substitute for human milk. Typically infant formulae are suitable to be used as sole source of nutrition. Such formulae are also known as starter formula. Formula for infants starting at 4 to 6 months, preferably 6 months of life to 12 months of life are intended to be supplementary feedings for infants that start weaning on other foods. Such formulae are also known as follow on formulae. Infant formulae and follow on formulae are subject to strict regulations, for example for the EU Commission Directive 2006/141/EC. In the present context, young child formula or growing up milk refers to nutritional compositions, artificially made, intended for infants of 12 months up to 48 months, or 1, 2 or 3 years of age, which are intended to be supplementary feedings to young children.

The nutritional composition according to the present invention is preferably an infant formula or a follow on formula or a young child formula, more preferably the nutritional composition is an infant formula or a follow on formula, even more preferably an infant formula.

The nutritional composition is preferably an infant formula or follow on formula and preferably comprises 3 to 7 g lipid per 100 kcal, preferably 4 to 6 g lipid per 100 kcal, more preferably 4.5 to 5.5 g lipid per 100 kcal, preferably comprises 1.6 to 3.5 g total protein per 100 kcal, preferably 1.8 to 2.4 g protein per 100 kcal, more preferably 1.85 to 2.0 g protein per 100 kcal, more preferably 1.8 to 2.0 g protein per 100 kcal and preferably comprises 5 to 20 g digestible carbohydrate per 100 kcal, preferably 7.5 to 15 g digestible carbohydrate per 100 kcal,

Preferably the nutritional composition is an infant formula or follow on formula, when ready to drink has an energy density of 60 kcal to 75 kcal per 100 ml, more preferably 60 to 70 kcal per 100 ml. This density ensures an optimal balance between hydration and caloric intake. The present nutritional composition is for use in providing nutrition to a child, preferably a young child, more preferably an infant.

In one embodiment, the nutritional composition is a powder. Suitably, the nutritional composition is in a powdered form, which can be reconstituted with water or other food grade aqueous liquid, to form a ready-to drink liquid, or is in a liquid concentrate form that should be diluted with water to a ready-to-drink liquid.

### Application

According to the present invention, upon feeding formulae comprising urea as well as at least one human milk oligosaccharide selected from 2′-FL, 3-FL, 3′-SL. 6′-SL, a urease positive B. infantis, a B. bifidum strain, and preferably further containing beta-galacto-oligosaccharides with a DP of 4 or more and a B. breve strain able to express to express beta1,4 endogalactanase a stimulating and/or synergistic effect on the growth of bifidobacteria, and fermentation activity of the bifidobacteria was observed. By releasing CO₂ and a nitrogen source by B. infantis subsequently a positive impact on the growth of bifidobacteria that are urease negative is anticipated. The released ammonia can be toxic for the host and therefore the subsequent consumption of ammonia by the bifidobacteria is beneficial. The urease negative B. bifidum released fucose that can be used by B. breve and B. infantis. The B. breve with beta1,4 endogalactanose is able to hydrolyse beta-galacto-oligosaccharides with DP of 4 or more, thereby making shorter beta-galacto-oligosaccharides available for B. infantis and B. bifidum. Therefore, the combination of the present invention further improves the microbiota and related health effects in infants or young children.

Thus in one embodiment, the present nutritional composition is for use in improving the intestinal microbiota in infants or young children, preferably infants, preferably for use in improving the intestinal microbiota in infants or young children by promoting the growth of bifidobacteria or by increasing the level of bifidobacteria. Also the diversity of the bifidobacteria may be increased.

The invention can also be worded as a method for improving the intestinal microbiota in infants or young children, preferably infants, preferably for use in improving the intestinal microbiota in infants or young children by promoting the growth of bifidobacteria or by increasing the level of bifidobacteria, the method comprising administering the present nutritional composition to said infants or young children.

In one embodiment improving the intestinal microbiota in infants or young children is considered to be non-therapeutic. Hence in one embodiment, the present invention concerns a non-therapeutic method for improving the intestinal microbiota in infants or young children, preferably infants, preferably for use in improving the intestinal microbiota in infants or young children by promoting the growth of bifidobacteria or by increasing the level of bifidobacteria, the non-therapeutic method comprising administering the present nutritional composition to said infants or young children. Likewise, the present invention can also be worded as the use of the present nutritional composition for improving the intestinal microbiota in infants or young children, preferably infants, preferably for improving the intestinal microbiota in infants or young children by promoting the growth of bifidobacteria or by increasing the level of bifidobacteria.

In one embodiment, the invention concerns the use of the present nutritional composition for increasing the level and/or metabolic activity of intestinal bifidobacteria, preferably in a child, preferably in an infant.

In the context of the present invention improving the intestinal microbiota or increasing the level of bifidobacteria means an higher number of the microbiota or bifidobacteria when compared to the number of microbiota or bifidobacteria in case one or more of a strain of Bifidobacterium longum ssp infantis able to express urease, a strain of Bifidobacterium bifidum able to express at least one extracellular enzyme selected from a fucosidase and a sialidase and not able to express a urease, 2′-fucosyllacose (2′-FL), 3-fucosyllactose (3-FL), 3′-sialyllactose (3′-SL), and 6′-sialyllactose (6′-SL) is not used.

Increasing the metabolic activity of intestinal bifidobacteria refers to the amount of acid produced by the intestinal bifidobacteria and an increasing is when compared to when one of the essential components as mentioned above is not use.

In one embodiment improving the intestinal microbiota in infants or young children, or improving the intestinal microbiota in infants or young children by promoting the growth of bifidobacteria, can be considered as being therapeutic, particularly in case the infants or young children are in need of having an improved intestinal microbiota, for example infants that are at risk of having compromised intestinal microbiota, or are having compromised intestinal microbiota. Thus in one embodiment, the present nutritional composition is for use in infants that are at risk of having compromised intestinal microbiota, or are having compromised intestinal microbiota. Such infants are for example infants born via C section, infants having allergy, preferably food allergy, or infants that are at risk of becoming allergic, preferably to food, preterm infants, infants that received antibiotics or receive human milk form mothers that are treated with antibiotics, infants that suffer or suffered from an infection, in particular an intestinal infection. Thus in one embodiment, the present nutritional composition is for use in improving the intestinal microbiota in infants born via C section, infants having allergy, preferably food allergy, infants that are at risk of becoming allergic, preferably to food, preterm infants, infants that received antibiotics, infants that suffer or have suffered from infection, in particular intestinal infection.

The invention can also be worded as a method for improving the intestinal microbiota in infants that are at risk of having compromised intestinal microbiota, or are having compromised intestinal microbiota, the method comprising administering the present nutritional composition to said infants. The invention can also be worded as a method for improving the intestinal microbiota in infants born via C section, infants having allergy, preferably food allergy, infants that are at risk of becoming allergic, preferably to food, preterm infants, infants that received antibiotics, infants that suffer or have suffered from infection, in particular intestinal infection, the method comprising administering the present nutritional composition to said infants.

In one embodiment, the present nutritional composition is for use in preventing or treating dysbiosis of the intestinal microbiota in infants or young children, preferably infants.

The invention can also be worded as a method for preventing or treating dysbiosis of the intestinal microbiota in infants or young children, preferably infants, the method comprising administering the present nutritional composition to said infants or young children.

In one embodiment, the present nutritional composition is for use in preventing and/or treating disorders associated with a compromised intestinal microbiota in infants or young children, preferably infants. In one embodiment, the present nutritional composition is for use in preventing and/or treating a disorder selected from the group consisting of diarrhea, constipation, intestinal inflammation, intestinal infection and allergy, preferably food allergy, in infants or young children, preferably infants.

In one embodiment, infants or young children with a compromised intestinal microbiota are infant or young children selected from the group consisting of a subject that was born preterm, a subject that was born via caesarean section, a subject that is or has been treated with antibiotics and a subject that is or has been at least partly breastfed by a woman taking antibiotics.

The invention can also be worded as a method for preventing and/or treating disorders associated with a compromised intestinal microbiota in infants or young children, preferably infants, the method comprising administering the present nutritional composition to said infants or young children. In one embodiment, the invention concerns a method for preventing and/or treating a disorder selected from the group consisting of diarrhea, constipation, intestinal inflammation, intestinal infection and allergy, preferably food allergy, in infants or young children, preferably infants, the method comprising administering the present nutritional composition to said infants or young children.

### EXAMPLES

### Example 1: Fermentation of 2′-Fucosyllactose by Single Strains and Mixes of Bifidobacterium Strains in the Presence or Absence of Urea as Nitrogen Source

### Materials and Methods

### Strains

The following Bifidobacterium strains were used. All strains were originally isolated from faeces of healthy infants.

| Species | Urease | Fucosidase | Obtained from |
|---|---|---|---|
| B. bifidum CNCM I-4319 | − | + (external) | Gervais Danone |
| B. breve C50 | − | − | Gervais Danone |
| B. infantis | + | + (internal) | ATCC 17930⁽¹⁾ |
| B. infantis BB-02 | + | + (internal) | Christian Hansen⁽²⁾ |
| | | | DSM333361 |

⁽¹⁾Used in example 3
⁽²⁾Used in examples 1 and 2

### Bifidobacterium Growth on 2′-FL

Bifidobacterium strains were anaerobically cultured at 37° C. on trans-galacto-oligosaccharide agar (TOS-Propionate Agar (Base), Merck, Darmstadt, Germany) supplemented with bifidobacterial selective component MUP (MUP selective supplement, HC735221, Merck kGaA, Darmstadt, Germany). Anaerobic conditions were applied by cultivation in an anaerobic cabinet with 90% N2, 5% H2, 5% CO2 atmosphere. Bifidobacterium strains were routinely streaked from their master seed lot (−80° C. stock) on TOS-Mup agar and were incubated anaerobically at 37° C. for two days.

The overnight pre-fermentations of the individual strains were performed anaerobically (95% N2, 5% CO₂ headspace) at 37° C. in the DASGIP Parallel Bioreactor Systems (DASGIP Information and Process Technology GmbH, Julich, Germany). pH-control (pH 6.5) was achieved by dosing 5M NaOH. The nitrogen-rich preculture medium with the following final composition was used:

1 g/l yeast extract, 15 g/l tryptone, 3 g/l KH2PO4, 4.8 g/l K2HPO4, 0.2 g/l MgS04, 0.5 g Cysteine·HCl, 15 g Sodium Propionate, supplemented with 5 ml vitamin mix, and 5 ml metal mix and 5 ml nucleotide precursor mix from concentrates (Teusink et al. 2005, AEM, Vol 7 1 (issue 11) p. 7253-7262) and 100 μM NiCl. The initial pH of this medium is 6.80 (+/−0.05). As carbon source 5 g/l lactose was used. For preculturing B. infantis 3 mM urea and 1 mM ammonium sulfate were added. For preculturing B. bifidum 8 mM ammonium sulfate was added. The medium was sterilized using filtration step with a 0.22 pm filter.

Fermentations with individual and multiple species were performed anaerobically (95% N2, 5% CO₂ headspace) at 37° C. in the DASGIP Parallel Bioreactor Systems (DASGIP Information and Process Technology GmbH, Julich, Germany). The working volume was 200 ml. pH-control (pH 6.5) was achieved by dosing 5M NaOH. The nitrogen-limited fermentation medium had the following final composition: 0.33 g/l yeast extract, 3 g/l KH2PO4, 1.87 g/l K2HPO4, 0.2 g/l MgS04, 0.2 g Cysteine·HCl, 10 g Sodium Propionate, supplemented with 3.33 ml vitamin mix, 3.33 ml metal mix and 10 ml nucleotide precursor mix from concentrates (Teusink et al. 2005, AEM, Vol 7 1 (issue 11) p. 7253-7262) and 100 μM NiCl. As carbon source 20 g/l 2′-fucosyllactose and 0.4 g/l lactose were used. When appropriate urea was added to a final concentration of 20 mg/100 ml. The pH of this medium is 6.50 (+/−0.05). The medium was sterilized using a filtration step with a 0.22 pm filter. For inoculation, the needed amount of bacterial cells was isolated from the calculated amount of precultures and isolated by centrifugation (10 min, 3000×g). After resuspension in a small volume (2 ml) of fermentation medium they were used to inoculate at an OD600 of 0.25 for each reactor. When 2 strains were inoculated the inoculation level was 0.125 for each strain.

Growth was monitored for 24 h and during the fermentations, samples were taken for further analysis like OD600, enumeration of Bifidobacteria, acid and ammonia production. The results are shown in Table 1.

TABLE 1
Growth, expressed as OD600, at 24 h of bifidobacteria
as single strain or mix, with 2-′FL as carbon
source, with or without urea as nitrogen source.

| | |
|---|---|
| OD600 at t = 24 h | OD600 AUC 0-24 h |
| N source | N source |

| | + urea | − | + urea | − |
|---|---|---|---|---|
| B. infantis | 2.3 | 1.3 | 32 | 21 |
| B. bifidum | 0.3 | 0.3 | 7 | 7 |
| mix | 3.7 | 1.3 | 41 | 20 |

As can be seen in Table 1 B. bifidum is not able to grow with urea as sole added nitrogen source. B. infantis shows an improved growth with urea as sole added nitrogen source. In the absence of urea, the mix of strains does not show an improved growth compared with B. infantis alone. Surprisingly, the in the presence of urea the combination of the two strains shows an improved effect on growth, which was 56% higher, based on final OD600, than the single B. infantis strain alone.

In Table 2 it is shown that base consumption, in other words acid production, is low in the absence of urea. Some base consumption is observed with the single strains, and even with B. bifidum as single strain some base consumption is observed. This is indicative for the B. bifidum strain being metabolically active, although not able to grow very well (see Table 1). The mix of strains in the absence of urea does not show an increased base consumption. Surprisingly in the presence of urea the base consumption is much higher, about 88%, than as can be expected based on the single B. infantis strain alone.

In Table 3 the formation of organic acids, the sum of acetate and lactate, which are the organic acids predominantly formed by the bifidobacterial, is shown. Acid formation is low in the absence of urea. Acid formation is observed with the single strains, and even with B. bifidum as single strain some acid production is observed. This is indicative for the B. bifidum strain being metabolically active. The mix of strains in the absence of urea does not show an increased acid production. Surprisingly in the presence of urea the acid production is much higher, about 57%, than can be expected based on the single B. infantis strain alone.

Ammonia production was observed in presence of urea and B. infantis only, as expected. In the mixture of B. infantis with B. bifidum, the measured ammonia levels were lower, indicating that the ammonia is consumed faster again by the B. infantis/B. bifidum consortium which in turn would be beneficial for the host. Results are shown in Table 4.

TABLE 2
NaOH consumption (mmol/l) at 24 h during growth of bifidobacteria
as single strain or mix with 2′-FL as carbon source,
with or without urea as nitrogen source.

| | |
|---|---|
| NaOH consumed at t = 24 h | AUC t 0-24 h |
| N source | N source |

| | + urea | − | + urea | − |
|---|---|---|---|---|
| B. infantis | 76 | 35 | 665 | 393 |
| B. bifidum | 19 | 19 | 258 | 255 |
| Mix | 143 | 37 | 1188 | 423 |

TABLE 3
Lactate + acetate production (mM) during growth
of bifidobacteria as single strain or mix with 2-′FL
as carbon source, with or without urea as nitrogen source.

N-source

| | + urea | − |
|---|---|---|
| B. infantis | 105.0 | 40.1 |
| B. bifidum | 23.5 | 25.7 |
| mix | 164.6 | 45.9 |

TABLE 4
Ammonia (mM) during growth of bifidobacterial
as single strain or mix with 2′-FL as carbon
source, with or without urea as nitrogen source

| N-source | |
|---|---|
| t = 9 h | t = 24 h |

| | + urea | − | + urea | − |
|---|---|---|---|---|
| B. infantis | 2.05 | nd | 0.42 | nd |
| B. bifidum | nd | 0.02 | nd | 0.01 |
| mix | 1.32 | 0.01 | nd | nd |

nd = not detectable

In Table 5 is shown that the total number of bifidobacteria is low in the absence of urea. With B. bifidum as single strain no increase in bifidobacteria is observed. With B. infantis as single strain bifidobacteria are higher. The mix of strains in the absence of urea does not show a relevant increase in bifidobacterial compared to B. infantis alone. Surprisingly in the presence of urea the number of bifidobacteria is much higher, more than twice as high than can be expected based on the single B. infantis strain alone.

TABLE 5
Total Bifidobacteria as cfu/ml × 1.10⁸ after 24 h growth
of bifidobacteria as single strain or mix with 2-′FL
as carbon source, with or without urea as nitrogen source.

N source

| | + urea | − |
|---|---|---|
| B. infantis | 5.1 | 1.6 |
| B. bifidum | 0.1 | 0.2 |
| mix | 11.0 | 1.9 |

### Example 2: Fermentation of 2-Fucosyllactose and Beta-Galacto-Oligosaccharides by Single Strains and Mixes of Bifidobacterium Strains in the Presence or Absence of Urea as Nitrogen Source

A similar experiment was performed as in example 1, except that as carbon source 2% bGOS//2′FL was used, in a 0.5:0.5 wt ratio. The bGOS used was VivinalGOS (FrieslandCampina), comprising 23.7 wt % lactose. For purpose of this in vitro experiment wherein the lactose is not digested, the lactose was considered as part of the bGOS.

In Table 6 it is shown that B. bifidum is not able to grow with urea as sole nitrogen source. B. infantis shows an improved growth with urea as sole nitrogen source. In the absence of urea, the mix of strains does not show an improved growth. Surprisingly, the combination of the two strains shows an improved effect on growth, which was about 30% higher than expected based on the results of the single B. infantis strain alone.

In Table 7 it is shown that base consumption is low in the absence of urea. With B. infantis as single strain some base consumption is observed, and even with B. bifidum as single strain some base consumption is observed. This is indicative for the B. bifidum strain being metabolically active even though this strain is not able to grow very well (Table 6). The mix of strains in the absence of urea does not show much increased base consumption. Surprisingly, in the presence of urea the base consumption by the mixture of strains is higher, about 38%, than can be expected based on the single B. infantis strain alone.

Table 8 shows the formation of organic acids, the sum of acetate and lactate, which are the organic acids predominantly formed by the bifidobacteria. Acid formation is low in the absence of urea. Some organic acid is formed by the B. infantis strain and even with B. bifidum as single strain some acid production is observed. This is indicative for the strain being metabolically active. The mix of strains in the absence of urea does not show much increased acid production. In the presence of urea, the acid production is higher than can be expected based on the single B. infantis strain alone.

Ammonia production was observed in presence of urea and B. infantis only, as expected. In combination with B. bifidum, the measured ammonia levels were lower, indicating that the ammonia is consumed faster again by the B. infantis/B. bifidum consortium which in turn would be beneficial for the host, see Table 9. Compared with 2′FL alone, in the presence of additional bGOS the ammonia produced from urea by the ureae does accumulate to a lesser extend in the media.

TABLE 6
Growth, expressed as OD600, at 24 h of bifidobacteria
as single strain or mix with bGOS/2-′FL as carbon
source, with or without urea as nitrogen source.

| | |
|---|---|
| OD600 t-24 h | OD600 AUC t 0-24 h |
| N source | N source |

| | + urea | − | + urea | − |
|---|---|---|---|---|
| B. infantis | 2.5 | 1.3 | 36 | 24 |
| B. bifidum | 0.3 | 0.3 | 7 | 7 |
| mix | 3.3 | 1.4 | 38 | 22 |

TABLE 7
NaOH consumption (mmol/l) at 24 h during growth of bifidobacteria
as single strain or mix with bGOS/2′-FL as carbon source,
with or without urea as nitrogen source.

| | |
|---|---|
| NaOH consumed at t = 24 h | AUC t 0-24 h |
| N source | N source |

| | + urea | − | + urea | − |
|---|---|---|---|---|
| B. infantis | 95 | 30 | 1008 | 393 |
| B. bifidum | 18 | 18 | 240 | 253 |
| mix | 133 | 43 | 1348 | 513 |

TABLE 8
Lactate + acetate production (mM) at 24 h during growth
of bifidobacteria as single strain or mix with 2-′FLand
bGOS/FOS as carbon source, with or without urea as nitrogen source.

N source

| | + urea | − |
|---|---|---|
| B. infantis | 132.1 | 43.7 |
| B. bifidum | 23.9 | 24.0 |
| mix | 150.0 | 55.3 |

TABLE 9
Ammonia (mM) during growth of bifidobacterial as
single strain or mix with 2′-FL and bGOS as carbon
source, with or without urea as nitrogen source

| N-source | |
|---|---|
| T = 9 h | T = 24 h |

| | + urea | − | + urea | − |
|---|---|---|---|---|
| B. infantis | 1.57 | nd | nd | nd |
| B. bifidum | nd | nd | nd | nd |
| mix | 1.17 | nd | nd | nd |

nd = not detectable

In Table 10 is shown that the total number of bifidobacteria is lower in the absence of urea. With B. bifidum as single strain no increase in bifidobacteria is observed. With B infantis bifidobacteria are high. The mix of strains in the absence of urea does not show a relevant increase in bifidobacterial compared to B. infantis alone. Surprisingly in the presence of urea the number of bifidobacterial is much higher, almost 2.5 times as high, than as can be expected based on the single B. infantis strain alone.

TABLE 10
Total Bifidobacteria as cfu/ml × 1.10⁸ after 24 h growth
of bifidobacteria as single strain or mix with 2-′FL and
bGOS/FOS as carbon source, with or without urea as nitrogen source.

N source

| | + urea | − |
|---|---|---|
| B. infantis | 6.6 | 1.3 |
| B. bifidum | 0.2 | 0.5 |
| mix | 16.0 | 1.8 |

### Example 3: Fermentation of HMOs Mixes, Beta-Galacto-Oligosaccharides by Single Strains and Mixes of Bifidobacterium Strains in the Presence or Absence of Urea as Nitrogen Source

A similar experiment was performed as in example 1, except that as carbon source 2% bGOS/FOS/HMOs was used, in a 0.45:0.05:0.5 wt ratio. The bGOS used was VivinalGOS (FrieslandCampina), comprising 23.7 wt % lactose. For purpose of this in vitro experiment wherein the lactose is not digested, the lactose was considered as part of the bGOS.

The total composition corrected for 100% CHO of the used mixture is: 5.3% FOS, 33.5% bGOS, 11.2% lactose, 2.5% glucose+galactose, 24.4% 2′-FL, 5.8% 3-FL, 13.0% LNT, 2.0% 3′-SL, 2.5% 6′-SL (100% total) The overnight pre-fermentations of the individual strains were performed anaerobically (90% N2, 5% H2, 5% CO₂ headspace) at 37° C. in the DASGIP Parallel Bioreactor Systems (DASGIP Information and Process Technology GmbH, Julich, Germany). pH-control (pH 6.5) was achieved by dosing 5M NaOH. The preculture medium with the following final composition was used: 0.33 g/l yeast extract, 3 g/l KH2PO4, 1.87 g/l K2HPO4, 0.2 g/l MgS04, 0.2 g Cysteine·—HCl, 10 g Sodium Propionate, supplemented with 3.33 ml vitamin mix, 3.33 ml metal mix and 10 ml nucleotide precursor mix from concentrates (Teusink et al. 2005, AEM, Vol 7 1 (issue 11) p. 7253-7262) and 100 μM NiCl. The initial pH of this medium is 6.50 (+/−0.05). As carbon source 5 g/l lactose was used. For preculturing B. infantis 10 mM urea and 2.2 mM ammonium sulfate were added. For preculturing B. breve and B. bifidum 10 mM ammonium sulfate was added. The medium was sterilized using filtration step with a 0.22 pm filter.

Fermentations with single and multiple species were performed anaerobically (90% N2, 5% H2, 5% CO2 headspace) at 37° C. in the DASGIP Parallel Bioreactor Systems (DASGIP Information and Process Technology GmbH, Julich, Germany). pH-control (pH 6.5) was achieved by dosing 5M NaOH. The nitrogen-limited fermentation medium had the following final composition: 0.33 g/l yeast extract, 3 g/l KH2PO4, 1.87 g/l K2HPO4, 0.2 g/l MgS04, 0.2 g Cysteine·—HCl, 10 g Sodium Propionate, supplemented with 3.33 ml vitamin mix, 3.33 ml metal mix and 10 ml nucleotide precursor mix from concentrates (Teusink et al. 2005, AEM, Vol 7 1 (issue 11) p. 7253-7262) and 100 μM NiCl. As carbon 20 g/l bGOS/FOS/HMO were used. When appropriate urea was added to a final concentration of 20 mg/100 ml. The pH of this medium is 6.50 (+/−0.05). The medium was sterilized using a filtration step with a 0.22 pm filter. For inoculation, the needed amount of preculture was directly added at an OD600 of 0.15 for each reactor. When multiple strains were inoculated the inoculation level was distributed equally for each strain.

Growth was monitored for 24 h and during the fermentations, samples were taken for further analysis like OD600, enumeration of Bifidobacteria and acid production.

### Glycoprofiling:

Samples of the culture supernatant were taken during the fermentation experiments. Samples for glycoprofiling taken during the fermentation were heated at 95° C. for 15 minutes to inactivate any enzymes and bacteria that were possibly still present. Samples were centrifuged for 10 min at 3250 g. After that, supernatants were filtered using a low-binding filter column (0.22 μm, MILLEX GV Durapore PVDF membrane, Merck, Darmstadt, Germany) and the samples were stored at −20° C. until analysis.

The glycoprofiling analysis was conducted using High Performance Anion Exchange Chromatography Pulsed Electrochemical Detection (HPAEC-PED) (Dionex-HPLC equipment ICS5000, Thermo Fisher Scientific Inc, Waltham, USA) where a PA200 column was used (Dionex™ CarboPac™ 4*250 nm, P/N 43055, Thermo Fisher Scientific Inc, Waltham, USA). The principle is that anion is exchanged where low pH gives carbohydrates a negative charge. Carbohydrates are then bond to the column after which elution takes place making use of an eluent with a low hydroxide concentration and sodium acetate gradient. Detection is done with Pulsed Electrochemical Detection. Sugars are oxidized at a gold electrode surface which causes a current which is detected. The gold electrode surface is cleaned and activated with a cyclic current pulse train which is specific for carbohydrate detection. Elution is in order of amount of charge but also size and shape play a role in the retention of the component. As internal standard arabinose was used. Methods to determine the glycoprofile are known in the art. An example is Finke et al, 2002, J. Agric. Food Chem. 2002, 50: 4743-4748.

### Results

Growth, expressed as OD600 at t=24 h is shown in Table 11. Table 11 shows the area under the curve (AUC) of the OD600. Growth of B. infantis or the mix comprising B. infantis is improved in the presence of urea, based on final OD at t=24. However, the area under the OD600 growth curve in the presence of urea is higher with urea and the mixture of bifidobacteria compared to B. infantis alone and this is indicative of a faster growth with the mix of strains.

TABLE 11
Growth, expressed as OD600, at 24 h of bifidobacteria
as single strain or mix, with bGOS/FOS/HMOs as carbon
source, with or without urea as nitrogen source.

| | |
|---|---|
| OD600 at t = 24 h | OD600 AUC 0-24 h |
| N source | N source |

| | + urea | − | + urea | − |
|---|---|---|---|---|
| B. breve | 1.8 | 2.1 | 28 | 29 |
| B. infantis | 3.5 | 1.2 | 35 | 17 |
| B. bifidum | 1.4 | 1.4 | 19 | 20 |
| mix | 3.3 | 2.4 | 45 | 30 |

Table 12 shows that base consumption was higher in the presence of urea for B. infantis. Base consumption was highest for the mix of bifidobacteria in the presence of urea and 36% higher than expected based on B. infantis strain alone. For the AUC value is 75% higher than based on B infantis alone, and this is indicative for a faster NaOH consumption as well.

TABLE 12
NaOH consumption (mmol/l) at 24 h during growth of bifidobacteria
as single strain or mix with bGOS/FOS/HMOs as carbon source,
with or without urea as nitrogen source.

| | |
|---|---|
| NaOH consumed T = 24 h | AUC 0-24 h |
| N source | N source |

| | + urea | − | + urea | − |
|---|---|---|---|---|
| B. breve | 108 | 108 | 1225 | 1200 |
| B. infantis | 138 | 63 | 1400 | 775 |
| B. bifidum | 43 | 38 | 550 | 450 |
| mix | 188 | 100 | 2450 | 975 |

In table 13 it is shown that the amount of organic acids formed (the sum of acetic acid and lactic acid) for B. infantis is higher in the presence of urea. Highest amount was observed with the mix of bifidobacteria and urea and the amount was higher than based on the B. infantis strain alone.

TABLE 13
Lactate + acetate production (mM) at 24 h during growth
of bifidobacteria as single strain or mix with bGOS/FOS/HMOs
as carbon source, with or without urea as nitrogen source.

N source

| | + urea | − |
|---|---|---|
| B. breve | 134.8 | 130.5 |
| B. infantis | 189.7 | 77.4 |
| B. bifidum | 50.7 | 112.0 |
| mix | 202.8 | 112.0 |

In Table 14 it is shown that the amount of bifidobacteria in the presence of urea is increased for B. infantis and decreased for B. bifidum. Highest amount of bifidobacteria was observed for the mix of bifidobacteria in the presence of urea. The amount was about 70% higher than based on the B. infantis strain alone.

TABLE 14
Total Bifidobacteria as cfu/ml × 1.10⁸ after 24 h growth
of bifidobacteria as single strain or mix with bGOS/FOS/HMOs
as carbon source, with or without urea as nitrogen source.

N source

| | + urea | − |
|---|---|---|
| B. breve | 2.3 | 2.3 |
| B. infantis | 6.6 | 1.0 |
| B. bifidum | 1.4 | 2.6 |
| mix | 11.1 | 4.8 |

Glycoprofiling the supernatants at timepoint 0, 3, 6, 9, 24 h revealed that there was little to no difference in carbohydrate consumption and the glycoprofile for B. breve or B. bifidum as single strains in the absence or presence of urea. The B. breve strain consumed the bGOS structures, while the B. bifidum showed poor growth with little consumption of bGOS. In table 15 only data for these two individual strains in the presence of urea is shown. For the B. infantis, a higher consumption of the different sugars was observed in the presence of urea. All carbohydrates except 3′-SL and 6′-SL (which were only partly consumed) were consumed at t=24 h in the presence of urea. 3′-SL and 6′-SL were consumed to a higher extent in the presence of urea.

### Mono- and Disaccharides:

Lactose and glucose were faster consumed in the presence of urea. Galactose is, by extracellular beta-galactosidase activity of B. bifidum, released into the medium, whereas for B. breve the excreted galactose was consumed again in time. With B. infantis galactose accumulates in the absence of urea but is hardly accumulated in the presence of urea. Fucose and neuraminic acid, building blocks of the HMOs, were released in the supernatants by B. bifidum extracellular alpha-L-fucosidase and sialidase activity, respectively, at 24 hours

The mix of 3 bifidobacteria in the presence of urea consumed glucose and lactose faster than the individual strains. Likewise, the mix of bifidobacteria shows accumulation of galactose in the absence of urea, but galactose is consumed in the presence of urea. Furthermore, fucose and neuraminic acid, were accumulated in the absence of urea, but did not accumulate in the presence of urea.

### GOS-Structures:

Two main bGOS structures were quantified, which were much faster consumed in the presence of urea. In addition to this a product from endogalactanase from B. breve, likely a beta1-4 galactotriose was formed when B. breve was present as single strain or as part of a mix. In the presence of urea this structure was completely consumed at t=24 h, whereas in the absence of urea this structure accumulates.

The mixture of bifidobacteria in the presence of urea consumed the beta1,4 and beta1,6 GOS, and faster than the individual strains, although the B. breve strain also consumed these carbohydrates fast. With the single strains little difference is observed in bGOS consumption in the presence or absence of urea. However, for the bifidobacteria mix the bGOS consumption is much faster in the presence of urea.

### HMO-Structures:

LNT is consumed by all three bifidobacteria strains with or without urea. The B. breve as single strain could not consume the fucosylated and sialyated HMOs, whereas the B. bifidum as single strain could consume these types of HMOs, but consumption was slow and the building blocks of the HMOs are accumulated in the growth medium (fucose, neuraminic acid and galactose). 2′-FL, 3-FL and 3′-SL and even 6′-SL were faster consumed in presence of urea. B. infantis metabolizes all five HMOs in the presence of urea, but hardly in the absence of urea.

With the bifidobacteria mix, metabolization of HMOs is much faster than B. infantis alone due to cross-feeding (syntrophic) interactions, in the absence as well as in the presence of urea, but being the highest in the presence of urea.

The results from the glycoprofiling analysis are indicative for a further improved syntrophic effects when to a mixture of B. infantis and B. bifidum and a HMO selected from 2′-FL, 3-FL, 3′-SL and 6′-SL further a B. breve with endogalactanase activity and bGOS that contains structures with DP of 4 or more is added.

TABLE 15
Urea effect on glycoprofile bifidobacterial mix; quantified peaks from glycoprofiles in mg/100 ml.

| | | | | | | | | | | | |
|---|---|---|---|---|---|---|---|---|---|---|---|
| lactose | glucose | galactose | 2′-FL | 3-FL | 3′-SL | 6′-SL | LNT | b1,4 GOS | b1,6 GOS | fucose | NeuAc |

B. breve + urea

| | | | | | | | | | | | | |
|---|---|---|---|---|---|---|---|---|---|---|---|---|
| t = 0 | 184 | 62 | 0 | 308 | 134 | 46 | 55 | 125 | 82 | 20 | 0 | 0 |
| t = 3 h | 117 | 47 | 20 | 311 | 125 | 44 | 53 | 132 | 81 | 16 | 0 | 0 |
| t = 6 h | 57 | 33 | 49 | 309 | 119 | 42 | 50 | 131 | 73 | 15 | 0 | 0 |
| t = 9 h | 20 | 20 | 82 | 313 | 125 | 42 | 51 | 130 | 68 | 15 | 0 | 0 |
| t = 24 h | 0 | 0 | 15 | 324 | 128 | 45 | 55 | 0 | 0 | 0 | 0 | 0 |

B. bifidum + urea

| | | | | | | | | | | | | |
|---|---|---|---|---|---|---|---|---|---|---|---|---|
| t = 0 | 184 | 62 | 0 | 308 | 134 | 46 | 55 | 125 | 82 | 20 | 0 | 0 |
| t = 3 h | 167 | 49 | 14 | 309 | 113 | 33 | 46 | 127 | 71 | 17 | 0 | 7 |
| t = 6 h | 178 | 40 | 31 | 296 | 113 | 28 | 47 | 115 | 72 | 17 | 7 | 10 |
| t = 9 h | 171 | 43 | 47 | 299 | 107 | 23 | 46 | 92 | 71 | 0 | 10 | 12 |
| t = 24 h | 55 | 37 | 135 | 281 | 98 | 0 | 42 | 6 | 69 | 0 | 26 | 21 |

B. infantis − no urea

| | | | | | | | | | | | | |
|---|---|---|---|---|---|---|---|---|---|---|---|---|
| t = 0 | 172 | 58 | 0 | 294 | 124 | 45 | 53 | 120 | 77 | 19 | 0 | 0 |
| t = 3 h | 152 | 56 | 7 | 258 | 119 | 41 | 48 | 113 | 76 | 19 | 0 | 0 |
| t = 6 h | 129 | 52 | 9 | 270 | 128 | 43 | 50 | 116 | 78 | 19 | 0 | 0 |
| t = 9 h | 103 | 46 | 11 | 301 | 123 | 41 | 48 | 129 | 77 | 17 | 0 | 0 |
| t = 24 h | 0 | 0 | 55 | 294 | 102 | 41 | 49 | 127 | 59 | 0 | 20 | 0 |

B. infantis + urea

| | | | | | | | | | | | | |
|---|---|---|---|---|---|---|---|---|---|---|---|---|
| t = 0 | 184 | 62 | 0 | 308 | 134 | 46 | 56 | 125 | 82 | 20 | 0 | 0 |
| t = 3 h | 164 | 56 | 6 | 342 | 128 | 44 | 53 | 141 | 83 | 20 | 0 | 0 |
| t = 6 h | 117 | 46 | 7 | 297 | 110 | 39 | 48 | 128 | 73 | 17 | 0 | 0 |
| t = 9 h | 82 | 38 | 9 | 247 | 114 | 33 | 40 | 123 | 65 | 12 | 0 | 0 |
| t = 24 h | 0 | 0 | 0 | 0 | 0 | 20 | 23 | 0 | 0 | 0 | 0 | 0 |

mix no urea

| | | | | | | | | | | | | |
|---|---|---|---|---|---|---|---|---|---|---|---|---|
| t = 0 | 172 | 58 | 0 | 294 | 124 | 45 | 53 | 120 | 77 | 19 | 0 | 0 |
| t = 3 h | 142 | 48 | 13 | 295 | 121 | 37 | 48 | 115 | 70 | 20 | 0 | 0 |
| t = 6 h | 103 | 28 | 23 | 251 | 121 | 31 | 42 | 106 | 66 | 13 | 0 | 0 |
| t = 9 h | 78 | 17 | 39 | 254 | 116 | 31 | 45 | 106 | 64 | 14 | 0 | 0 |
| t = 24 h | 7 | 0 | 103 | 247 | 29 | 0 | 33 | 0 | 39 | 0 | 69 | 7 |

mix + urea

| | | | | | | | | | | | | |
|---|---|---|---|---|---|---|---|---|---|---|---|---|
| t = 0 | 184 | 62 | 0 | 308 | 134 | 46 | 55 | 125 | 82 | 20 | 0 | 0 |
| t = 3 h | 137 | 49 | 12 | 267 | 125 | 34 | 45 | 114 | 70 | 16 | 0 | 0 |
| t = 6 h | 80 | 11 | 21 | 243 | 118 | 20 | 42 | 106 | 64 | 0 | 0 | 0 |
| t = 9 h | 15 | 0 | 29 | 25 | 117 | 27 | 41 | 102 | 60 | 0 | 0 | 0 |
| t = 24 h | 0 | 0 | 0 | 0 | 0 | 0 | 3 | 0 | 0 | 0 | 0 | 0 |

### Example 4: Fermentation of 2′-Fucosyllactose by Single Strains and Mixes of Bifidobacterium Strains: Antagonism and Synergism in Growth with Different Mixes

Growth experiments were performed as in example 1, 2 and 3, except that instead of urea ammonia was used.

These experiments are not part of the invention but demonstrate that B. breve together with B. infantis show antagonism of growth and acid production, which is alleviated in the presence of a B. bifidum strain.

### Results

The results for NaOH consumption are shown in Table 16.

TABLE 16
NaOH consumed at t = 24 h upon fermentation of 2′-FL
in BASE-Mup medium by single strains or mixes of strains. For
a mix of strains the ‘Relative NaOH consumption. vs single
strain (%)’ is compared to the best performing single strain of that mix.

| B. | B. | B. breve | NaOH | Relative NaOH cons. |
|---|---|---|---|---|
| infantis | bifidum | C50 | (mmol/l) | vs single strain (%) |
| X | | | 278 | 100 |
| | X | | 232 | 100 |
| | | X | 0 | 0 |
| X | X | | 273 | 98 ¹⁾ |
| X | | X | 209 | 76 ²⁾ |
| X | X | X | 279 | 100 ³⁾ |

¹⁾ No synergy on total NaOH consumption;
²⁾ Antagonism on NaOH consumption;
³⁾ No antagonism anymore

Based on the NaOH consumption at t=24 h (indicative for total acid produced upon fermentation) it can be concluded that B. infantis and B. bifidum as single strain could ferment 2′-FL and the B. breve strain could not ferment 2′-FL as single strain.

The combination of B. infantis and B. bifidum did not show an improved effect on NaOH consumption compared to the single strains. in accordance with example 1 in the absence of urea.

When a combination of B. infantis with B. breve was tested, the total amount of NaOH consumed with the mix was 25% decreased compared to the single B. infantis strain, indicative of an antagonistic effect. Interestingly, when a mix of B. infantis, B. breve and B. bifidum was tested, an NaOH consumption was observed which was similar to the B. infantis strain alone (which had a higher NaOH consumption that the B. bifidum strain alone) and this indicates that the antagonistic effect between B. breve and B. infantis has been alleviated when B. bifidum is also present in the mix.

Besides the total amount of NaOH consumed, the rate of NaOH consumption during the logarithmic growth phase was reduced in the combination of B. breve and B. infantis compared to the B. infantis strain alone (data not shown). The rate of NaOH consumption was slightly increased in the mix of B. breve, B. infantis and B. bifidum, indicating that not only the antagonistic effect between B. breve and B. infantis was alleviated, but even a small synergistic effect was observed (data not shown).

### Example 5

An infant formula comprising per 100 ml (after reconstitution of 13.6 g of powder with 90 ml of water):
65 kcal
1.345 g total protein (based on total N*6.25) whey protein/casein in 6/4 wt ratio and including 25 mg urea
6.9 g lactose
3.36 g fat (mixture of vegetable oil, fish oil, comprising LA, ALA, DHA and ARA)
0.8 g non digestible oligosaccharides
0.8 g (bGOS/IcFOS in a 9:1 ratio)
0.1 g 2′-FL (Chr Hansen)
probiotics, being present at 10⁶-10⁹ cfu/g powder and comprising:
   B. breve C50
   B. infantis BB-02
   B. bifidum CNCM I-4319
minerals, trace elements, vitamins and other micronutrients as known in the art and according to international guidelines for infant formula; additional NPN are 2.2 mg nucleotides, 1.6 mg carnitine, 12 mg choline and 5.3 mg taurine.

### Example 6

A follow on formula comprising per 100 ml (to be reconstituted from 14.0 g of powder with 90 ml of water):
69 kcal
1.4 g total protein (based on total N*6.25) whey protein/casein in 6/4 wt ratio and including 20 mg urea
8.2 g lactose
3.2 g fat (mixture of vegetable oil, fish oil, comprising LA, ALA, DHA and ARA)
0.8 g non digestible oligosaccharides
   (bGOS/IcFOS in a 9:1 ratio)
   0.11 g 2′-FL (Chr Hansen)
probiotics, being present at 10⁵-10⁹ cfu/g powder and comprising:
   B. breve C50
   B. infantis BB-02
   B. bifidum CNCM 14319
minerals, trace elements, vitamins and other micronutrients as known in the art and according to international guidelines for infant formula; additional NPN are 2.2 mg nucleotides, 1.6 mg carnitine, 12 mg choline and 5.3 mg taurine.

### Example 7

Young child formula, in powder form, comprising per 100 kcal:
1.8 g protein (whey protein:casein 4:6) including 20 mg urea
4.0 g lipid (mainly vegetable lipid)
13.4 g digestible carbohydrate (mainly lactose)
non digestible oligosaccharides:
   1 g bGOS (coming from VivinalGOS)
   0.11 g 3′-SL (GeneChem)
probiotics, being present at 10⁵-10⁹ cfu/g powder and comprising:
   B. infantis M-63
   B. bifidum CNCM 1-4319
minerals, vitamins and other micronutrients as known in the art

The powder is in a pack that contains instructions to dilute with water and the ready to drink formula has 65 kcal/100 ml.

1. A nutritional composition comprising urea, non-digestible oligosaccharide, and lactic acid producing bacteria, wherein the non-digestible oligosaccharide comprises one or more human milk oligosaccharides selected from the group consisting of 2′-fucosyllacose (2′-FL), 3-fucosyllactose (3 FL), 3′-sialyllactose (3′-SL), and 6′-sialyllactose (6′-SL), and wherein the lactic acid producing bacteria comprise a strain of Bifidobacterium longum ssp infantis able to express urease, and a strain of Bifidobacterium bifidum able to express at least one extracellular enzyme selected from a fucosidase and a sialidase and not able to express a urease.

2. The nutritional composition according to claim 1, comprising 2′-fucosyllactose.

3. The nutritional composition according to claim 1, wherein the non-digestible oligosaccharide further comprises beta-galacto-oligosaccharides comprising beta1,4 linkages and having a degree of polymerization of at least 4.

4. The nutritional composition according to claim 1, which further comprises a strain of Bifidobacterium breve able to express an extracellular beta1,4 endogalactanase.

5. The nutritional composition according to claim 1, wherein the non-digestible oligosaccharide further comprises non-digestible polyfructose.

6. The nutritional composition according to claim 1, comprising at least four human milk oligosaccharides selected from the group consisting of 2′ fucosyllactose, 3-fucosyllactose, 3′-sialyllactose, 6′-sialyllactose, lacto N tetraose, lacto N neotetraose and difucosyllactose.

7. The nutritional composition according to claim 1, wherein the urea is present in an amount of at least 15 mg per 100 ml.

8. The nutritional composition according to claim 1, further comprising protein, digestible carbohydrate and lipid.

9. The nutritional composition according to claim 8 which is an infant formula, a follow on formula or a young child formula.

10. The nutritional composition according to claim 8, wherein the urea is present at least 1.13 wt %.

11. The nutritional composition according to claim 8, comprising 1.0 to 2.1 g protein per 100 kcal.

12. The nutritional composition according to claim 8, comprising at least 90 wt % cow's milk proteins based on total protein.

13. The nutritional composition according to claim 1, comprising at least 0.01 g/100 ml human milk oligosaccharide.

14. The nutritional composition according to claim 3, wherein the amount of galacto-oligosaccharide comprising beta1,4 linkages having a DP of at least 4 is at least 0.05 mg/100 ml, and/or 0.35 wt % based on dry weight of the composition.

15. The nutritional composition according to claim 1, wherein the non-digestible oligosaccharides are present an amount of at least 1.5 wt, based on dry weight of the composition, and/or at least 0.2 g per 100 ml.

16. The nutritional composition according to claim 1, that contains at least 10⁵ cfu B. longum ssp infantis able to express a urease, per gram dry weight of the nutritional composition and at least 10⁵ cfu B. bifidum able to express at least one extracellular enzyme selected from a fucosidase and a sialidase and not to able to express a urease, and optionally at least 10⁵ cfu B. breve able to express beta1,4 endogalactanase, per gram dry weight of the nutritional composition, and wherein the total amount of bifidobacteria is at least 10⁶ cfu per gram dry weight of the nutritional composition.

17.-21. (canceled)

22. A method for increasing the level and/or metabolic activity of intestinal bifidobacteria, in a subject, preferably in a child, preferably in an infant, the method comprising administering the composition of claim 1.

23. A method for preventing and/or treating intestinal microbial dysbiosis and/or for reducing intestinal pathogenic bacteria and/or for preventing and/or treating a disorder associated with a compromised intestinal microbiota in infants or young children subject, preferably in a child, preferably in an infant, the method comprising administering the composition of claim 1.

24. The method according to claim 23, wherein the subject is a human subject suffering from or being at risk of intestinal microbial dysbiosis.

25. The method according to claim 24, wherein the disorder is selected from the group consisting of diarrhea, constipation, intestinal inflammation, intestinal infection and allergy.
